(19) United States
(12) Patent Application Publication
(10) Pub. No.: US 2009/0177123 A1
(43) Pub. Date: Jul. 9, 2009

(54) Methods for treating inflammatory disorders

(75) Inventor: Michael T. Peterson, Lakeville, MN (US)

Correspondence Address: ROPES & GRAY LLP, PATENT DOCKETING 39/41, ONE INTERNATIONAL PLACE, BOSTON, MA, 02110-2624, US

(73) Assignee: Celleration, Inc., Eden Prairie, MN (US)

(21) Appl. No.: 12/317,710

(22) Filed: Dec. 23, 2008

Related U.S. Application Data
(60) Provisional application No. 61/009,348, filed on Dec. 28, 2007.

(51) Int. Cl. A61N 7/00; A61M 35/00

(52) U.S. Cl. 601/2; 604/20; 607/96; 128/898

(57) ABSTRACT

The invention provides methods for treating inflammatory disorders by administering low frequency ultrasound energy to decrease the inflammatory response. Exemplary inflammatory disorders are rheumatoid disorders including, but not limited to, rheumatoid arthritis, juvenile arthritis, bursitis, gout, spondylitis, scleroderma, Still's disease, and vasculitis.

### RELATED APPLICATIONS

This application claims the benefit of priority to U.S. provisional application No. 61/009,348, filed Dec. 28, 2007. The specification of the foregoing application is hereby incorporated by reference in its entirety.

### BACKGROUND

Inflammatory disorders, such as rheumatoid disorders, have a substantial impact on the quality of life of sufferers. These conditions can be physically uncomfortable, or even debilitating. Rheumatoid disorders are often progressive and degenerative, and thus patients slowly experience the decline in their ability to perform every day tasks.

Current therapies for inflammatory disorders include antibiotics, steroids, and immunosuppressive agents. These treatments are typically delivered systemically. Although such therapies may provide improvement for some patients, they may also have side-effects that limit their utility. For example, long term antibiotic therapy may promote the emergence of antibiotic resistant strains of bacteria. Steroids and other immunosuppressants can place patients at increased risk for infection. Additionally, steroid therapy itself may have undesirable effects on appearance by causing weight gain, blotting, and puffiness.

Ultrasound has been used in a variety of diagnostic and therapeutic contexts. High frequency ultrasound energy has been used in diagnostic imaging and lithotripsy. Low frequency ultrasound has been used in wound debridement and to promote the healing of serious wounds. Some applications of low frequency ultrasound rely on contact between the tissue and the ultrasound transducer tip or sonotrode (See, for example, technology used by Soring and Misonix; www.soring.com; www.misonix.com). Other applications of low frequency ultrasound deliver therapeutically effective doses of energy without contact between patient tissue and the ultrasound transducer tip (See, for example, technology developed by Celleration, Inc., www.celleration.com). By avoiding contact with patient tissue, non-contact ultrasound devices and methods are particularly well suited for treating painful wound tissue.

### SUMMARY

Despite numerous uses for ultrasound energy that exist in the art, low frequency, non-contact ultrasound has not been used to provide a safe and effective treatment for inflammatory disorders.

The present invention provides methods and devices for treating inflammatory disorders using low frequency ultrasound delivered without contact between the ultrasound transducer tip, or other component of the device, and the tissue to be treated. The method is of particular use in the treatment of rheumatoid disorders. Exemplary rheumatoid disorders that can be treated include, but are not limited to, rheumatoid arthritis, juvenile arthritis, bursitis, spondylitis, gout, scleroderma, Still's disease, and vasculitis.

The present invention is based in part on the demonstration that low frequency ultrasound energy delivered at a non-contact distance decreases expression of factors indicative of the inflammatory response. For example, low frequency, non-contact ultrasound treatment decreases both the expression of TNF-α and the activation of p38 MAPK, without affecting the viability of cells of the immune system. The ability of low frequency ultrasound to specifically modulate the inflammatory response, combined with the ease in delivering low frequency ultrasound locally to an effected tissue, makes it especially well suited for use in the treatment of inflammatory disorders. Low frequency ultrasound is particularly well suited for use in the treatment of rheumatoid disorders. Accordingly, the present invention provides methods for treating inflammatory disorders including, but not limited to, rheumatoid disorders.

In a first aspect, the present invention provides a method for treating an inflammatory disorder by delivering low frequency ultrasound energy from a non-contact distance to effected tissue of a patient in need thereof. The ultrasound energy penetrates the skin to provide a therapeutic effect to the skin and/or to the underlying tissue. For example, the method results in a decrease in the inflammatory response. Over the course of one or more treatments, the use of low frequency ultrasound results in a reduction or elimination of one or more of the symptoms of the inflammatory disorder. Additionally, over the course of one or more treatments, the use of low frequency ultrasound results in a decrease of the frequency and/or severity of symptoms or outbreaks of symptoms.

In certain embodiments, the inflammatory disorder is a rheumatoid disorder. Rheumatoid disorders are inflammatory disorders of the connective tissue (e.g., joints, blood vessels, tendons, ligaments, etc.). Methods of treating a rheumatoid disorder comprise delivering ultrasound energy at a non-contact distance from the skin overlying the effected tissue. Thus, for example, if a symptom of the rheumatoid disorder is inflammation and pain in the joints of the fingers, the instant method comprises delivering low frequency ultrasound at a non-contact distance from the skin overlying the joints of the fingers. The energy penetrates the skin to provide a therapeutic effect to the underlying joints. Exemplary rheumatoid disorders include, but are not limited to, rheumatoid arthritis, juvenile arthritis, bursitis, gout, spondylitis, scleroderma, Still's disease, and vasculitis.

In certain embodiments, the ultrasound energy is delivered via a liquid spray, and the method comprises delivering low frequency ultrasound energy and a liquid spray from a non-contact distance to effected tissue of a patient in need thereof. Exemplary liquids include, but are not limited to, saline and water. Optionally, the liquid can include a medicament such as an antibiotic, an astringent, an anti-inflammatory, a steroid, or an analgesic. In certain embodiments, the medicament is a rheumatoid disorder-specific medicament, for example, a TNFα antagonist. In other embodiments, the liquid can include a moisturizer, skin conditioner, vitamins, or minerals. In other embodiments, the liquid consists essentially of saline or water, and does not include a medicament. However, liquids that do not contain medicament can contain preservatives to improve their shelf life, or other inert agents that are not designed to have an effect on patient tissue.

In certain embodiments, the ultrasound energy is delivered without a liquid spray. In other words, the method comprises delivering ultrasound energy from a non-contact distance and in the absence of a liquid spray or coupling agent.

In certain embodiments, the ultrasound energy acts, in part, to facilitate delivery of drug to patient tissue. For example, a medicament is delivered to the ultrasound transducer to create a spray, and ultrasound energy and the spray are delivered to the patient tissue. In other embodiments, the medicament is applied topically directly to skin overlying patient tissue in a first step, and ultrasound energy is then delivered to the topically applied medicament and the patient tissue. When used in this manner, ultrasound energy can be used “dry” or “wet” to facilitate penetration of both the topically applied medicament and the ultrasound energy.

In certain embodiments, the method for treating an inflammatory disorder comprises multiple treatments. For example, patients may receive doses of ultrasound two or more times per week, for one, two, three, four, or more than four weeks. Alternatively, patients may receive daily doses of ultrasound energy (daily treatments). In certain embodiments, the method comprises a single treatment.

In certain embodiments, the duration and/or frequency of treatment is varied over time depending on the severity of the patient's condition. For example, a patient who presents with severe symptoms may be initially treated daily. As the patient's symptoms decrease in severity, the frequency of treatment may be decreased to, for example, three treatments/week. As the symptoms completely subside, treatment may be discontinued entirely. Alternatively, the patient, particularly a patient at risk for recurrence of symptoms or a patient whose condition is chronic, may be placed on a prophylactic regimen of, for example, weekly treatments intended to help prevent or delay recurrence of symptoms (or, to decrease the severity of recurring symptoms).

The appropriate number of treatments, and the duration of each treatment, can be determined by a health care provider based on, for example, the particular inflammatory disorder being treated, the severity of the disorder, and the overall health of the patient.

In certain embodiments, each treatment comprises delivering ultrasonic energy to patient tissue for at least approximately thirty consecutive seconds. In certain embodiments, each treatment comprises delivering ultrasonic energy to patient tissue for at least approximately 1 minute, at least approximately 2 minutes, at least approximately 3 minutes, at least approximately 4 minutes, or at least approximately 5 minutes. In certain embodiments, each treatment comprises delivering ultrasonic energy to patient tissue for at least approximately 6 minutes, at least approximately 7 minutes, at least approximately 8 minutes, at least approximately 9 minutes, or at least approximately 10 minutes. In other embodiments, each treatment comprises delivering ultrasonic energy for approximately 5-10 minutes, approximately 10-15 minutes, or approximately 15-20 minutes.

In certain embodiments, the therapeutic effect includes decreasing an inflammatory response, as assayed by expression of TNF-α or other inflammatory cytokine. Therapeutic efficacy also includes one or more of decreasing bacterial count, increasing healing, decreasing pain, decreasing swelling, and improving range of motion. Over the course of therapy, therapeutic efficacy can be assessed by evaluating improvement, such as a decrease in the presence or severity of the symptoms of the inflammatory disorder.

In certain embodiments, the low frequency ultrasound energy delivered is approximately 10-100 kHz, approximately 20-80 kHz, approximately 20-40 kHz, approximately 35-60 kHz, or approximately, 40-50 kHz. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

In certain embodiments, the low frequency ultrasound energy is also low intensity ultrasound energy. Intensity refers to the amount of energy transferred to the tissue. In certain embodiments, the low frequency, low intensity energy has an intensity of approximately 0.1 to 2.2 W/cm². In certain embodiments, the low frequency, low intensity energy has an intensity of approximately 0.1 to 0.75 W/cm². In certain embodiments, the low frequency, low intensity energy has an intensity of approximately 0.4-0.7 W/cm². In certain embodiments, the low frequency, low intensity energy has an intensity of approximately 0.5 W/cm².

In certain embodiments, separation distance (non-contact distance) between the distal most surface of the ultrasound therapy device and the skin overlying the patient tissue being treated is a non-contact distance of at least 0.1 inches (2.5 mm). Preferably, the separation distance is from about 2.5 mm to about 51 cm, more preferably, from about 15 mm to about 25 mm. Regardless of the exact distance, non-contact treatment means that there is no contact between the ultrasound device and the skin overlying tissue that is being treated. If the skin is the tissue being treated, than the non-contact distance is understood to be the distance between the distal most surface of the device and the skin being treated.

In certain embodiments, the low frequency ultrasound treatment does not result in a significant increase in the temperature of the skin overlying the tissue being treated.

In certain embodiments, the low frequency ultrasound treatment does not result in a significant decrease in viability of human cells in the treated tissue.

In certain embodiments, the low frequency ultrasound treatment decreases the symptoms of the inflammatory disorder and promotes healing of the effected tissue without significant scarring.

In certain embodiments, low frequency ultrasound therapy is part of a therapeutic regimen used in conjunction with one or more additional treatment modalities. For example, a patient may also receive topical or oral medications, or local or systemic injections. In certain embodiments, treatment with low frequency ultrasound therapy decreases the dosage or frequency of medication used in comparison to that used in the absence of ultrasound therapy. Additionally, patients may also use diet, acupuncture, stress management, exercise, physical therapy, occupational therapy, or other herbal or homeopathic therapies to help manage the symptoms of their inflammatory disorder.

In another aspect, the invention provides a method for reducing pain associated with a rheumatoid disorder in a patient in need thereof. The method comprises providing a transducer which can emit low frequency ultrasonic energy. Ultrasonic energy is delivered to effected tissue of said patient. The energy is delivered from a non-contact distance between a vibrating tip of the transducer and the effected tissue of said patient. The delivered ultrasonic energy provides a therapeutic effect to reduce pain associated with the rheumatoid disorder.

In certain embodiments, the effected tissue treated is selected from one or more of joints, tendons, and ligaments.

In certain embodiments, the ultrasonic energy is delivered via a liquid spray, and the method comprises delivering the low frequency ultrasonic energy and the liquid spray to said patient. In other embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the effected tissue or administering a systemic medicament prior to and/or following delivering said ultrasonic energy. In certain embodiments, the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

In certain embodiments, the step of delivering ultrasonic energy comprises delivering ultrasonic energy for at least about 2 consecutive minutes (e.g., this defines one treatment). In certain embodiments, the method comprises delivering ultrasonic energy at least twice per week (e.g., at least two treatments per week) for at least two weeks.

In certain embodiments, the rheumatoid disorder is rheumatoid arthritis or juvenile arthritis. In certain embodiments, the rheumatoid disorder is selected from any of bursitis, spondylitis, gout, scleroderma, Still's disease, and vasculitis.

In another aspect, the invention provides a method for treating a rheumatoid disorder. The method comprises providing a transducer which can emit low frequency ultrasonic energy and delivering said ultrasonic energy to a patient in need thereof. The ultrasonic energy is delivered from a non-contact distance between a vibrating tip of the transducer and treated patient tissue, and the delivered ultrasonic energy provides a therapeutic effect to decrease one or more symptoms of the rheumatoid disorder in said patient.

In certain embodiments, the ultrasonic energy is delivered to at least one of said patient's joints, ligaments, or tendons.

In certain embodiments, the ultrasonic energy penetrates the patient tissue to provide a therapeutic effect.

In certain embodiments, the ultrasonic energy is delivered via a liquid spray, and the method comprises delivering the low frequency ultrasonic energy and the liquid spray to the patient. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the treated tissue or systemically administering medicament prior to and/or following delivering said ultrasonic energy. In certain embodiments, the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

In certain embodiments, delivering ultrasonic energy comprises delivering ultrasonic energy for at least about 2 consecutive minutes (e.g., this defines one treatment). In certain embodiments, delivering ultrasonic energy comprises delivering ultrasonic energy at least twice per week for at least two weeks.

In certain embodiments, the one or more symptoms are selected from one or more of inflammation, pain, tingling, weakness, decreased grip strength, swelling, itchiness, burning, fever, and decreased range of motion.

In certain embodiments, the rheumatoid disorder is rheumatoid arthritis or juvenile arthritis. In other embodiments, the rheumatoid disorder is selected from any of bursitis, spondylitis, gout, scleroderma, Still's disease, and vasculitis.

In another aspect, the invention provides a method for managing symptoms of a rheumatoid disorder. The method comprises providing a transducer which can emit low frequency ultrasonic energy and delivering said ultrasonic energy to a patient in need thereof for at least two consecutive minutes at least twice per week (e.g., providing at least 2 treatments/week). The ultrasonic energy is delivered from a non-contact distance between a vibrating tip of the transducer and treated patient tissue, and the delivered ultrasonic energy provides a therapeutic effect to treat one or more symptoms of the rheumatoid disorder.

In another aspect, the invention provides a method for decreasing the number of painful or swollen joints in a patient suffering from a rheumatoid disorder. The method comprises providing a transducer which can emit low frequency ultrasonic energy and delivering said ultrasonic energy to said patient. The ultrasonic energy is delivered from a non-contact distance between a vibrating tip of the transducer and treated patient tissue, and the delivered ultrasonic energy provides a therapeutic effect to decrease the number of painful or swollen joints in said patient.

In another aspect, the invention provides a method for decreasing expression of an inflammatory cytokine in a patient having a rheumatoid disorder. The method comprises providing a transducer which can emit low frequency ultrasonic energy and delivering said ultrasonic energy to said patient in need thereof. The delivered ultrasonic energy decreases expression of said inflammatory cytokine in one or more of an effected joint, ligament, tendon, skin, or blood vessel of said patient, and the inflammatory cytokine is TNFα.

In certain embodiments, the ultrasonic energy is delivered via a liquid spray, and the method comprises delivering the low frequency ultrasonic energy and the liquid spray to the patient. In certain embodiments, the ultrasonic energy is delivered in the absence of a liquid spray or coupling agent.

In another aspect, the invention provides a method for decreasing and/or assessing expression of TNF-α in response to a stimulus. In certain embodiments, the method is an in vitro method used to modulate the expression of TNF-α in cells in vitro. In other words, cells or tissue explants cultured or maintained in vitro (outside the context of a person or whole organism) can be contacted with ultrasound energy and monitored to assess expression of TNF-α. When used in this manner, the invention provides an in vitro diagnostic method for decreasing or evaluating TNF-α expression in cells or tissue explants maintained in culture. In certain other embodiments, the method is an in vivo method. In other words, the invention provides a diagnostic method whereby TNF-α expression is monitored following in vivo treatment with ultrasound energy. Note that in vivo and in vitro refer to the status of the cells at the time the ultrasound energy is delivered. However, the assessment of TNF-α expression can occur either within or outside the context of the organism.

In certain embodiments, TNF-α expression is assessed in one or more cell types of the immune system, such as activated monocytes or macrophages. In certain embodiments, the stimulus is an allergen or irritant.

In certain embodiments, the method is used to assess the progress or effectiveness of the treatment of an inflammatory disease, such as a rheumatoid disorder. When used in this manner, assaying the expression of TNF-α or another pro-inflammatory cytokine can be used as a diagnostic to monitor improvement of the patient over the course of one or more treatments. This diagnostic step can be performed at about the same time as a therapy (just before or just after therapy). Alternatively, the diagnostic step can be performed at a different time, such as during a non-therapy day between treatments.

In another aspect, the invention provides a method for modulating the expression of one or more inflammatory cytokines in response to a stimulus. In certain embodiments, the method is an in vivo method. In certain embodiments, the method is an in vitro method. In certain embodiments, the in vitro or in vivo method is used to modulate the expression of TNF-α, the activation of p38, or the expression of one or more interleukins.

In certain embodiments, expression or activation of an inflammatory cytokine is assessed in one or more cell types of the immune system, such as activated monocytes or macrophages. In certain embodiments, the stimulus is an allergen or irritant.

In another aspect, the invention provides a method of drug delivery. Ultrasound energy can be used to deliver medicament to effected patient tissue, thereby treating an inflammatory disorder, for example, a rheumatoid disorder.

In certain embodiments, the medicament is formulated for delivery as a liquid spray. When delivered as a spray, the liquid contacts the ultrasound transducer, thereby generating a liquid spray. The liquid spray and ultrasound energy are delivered to the effected tissue from a non-contact distance.

In other embodiments, the medicament is applied topically, directly to the effected tissue or to the skin overlying the effected tissue. Ultrasound energy is then delivered to the medicament and to the patient tissue. Without being bound by theory, the ultrasound energy facilitates the penetration of the topically applied medicament, and both the medicament and the ultrasound energy penetrate the tissue.

When ultrasound energy is used to facilitate drug delivery, medicament can be delivered more quickly and in a more targeted fashion. Additionally, given the improved tissue penetration, the use of ultrasound energy can help decrease the dosage of medicament required for therapeutic efficacy. This is particularly advantageous when administering drugs with potentially harmful side-effects, or when administering drugs that are very expensive.

The invention contemplates combinations of one or more of any of the foregoing or following aspects and embodiments of the invention.

### DETAILED DESCRIPTION OF THE DRAWINGS

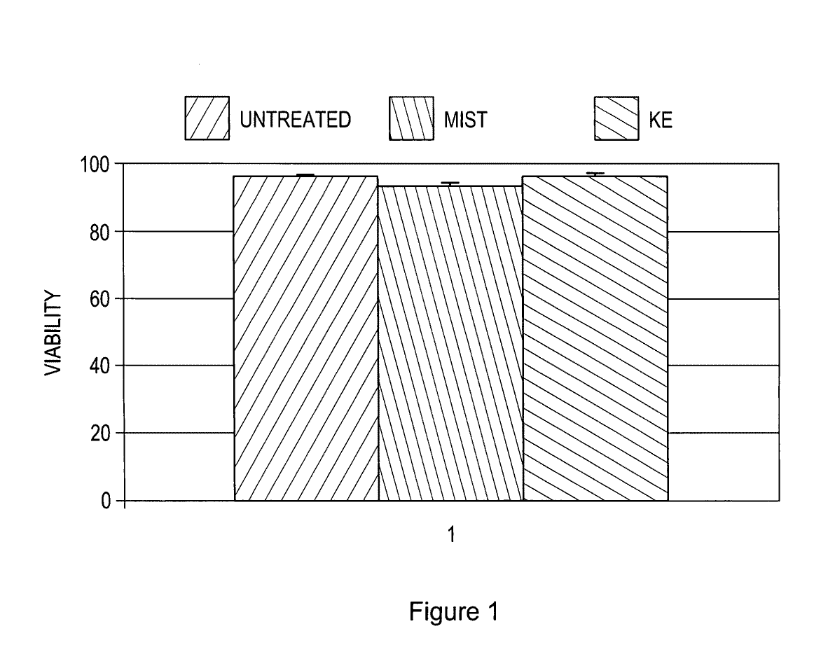

FIG. 1 shows that low frequency ultrasound energy does not significantly affect cell viability.

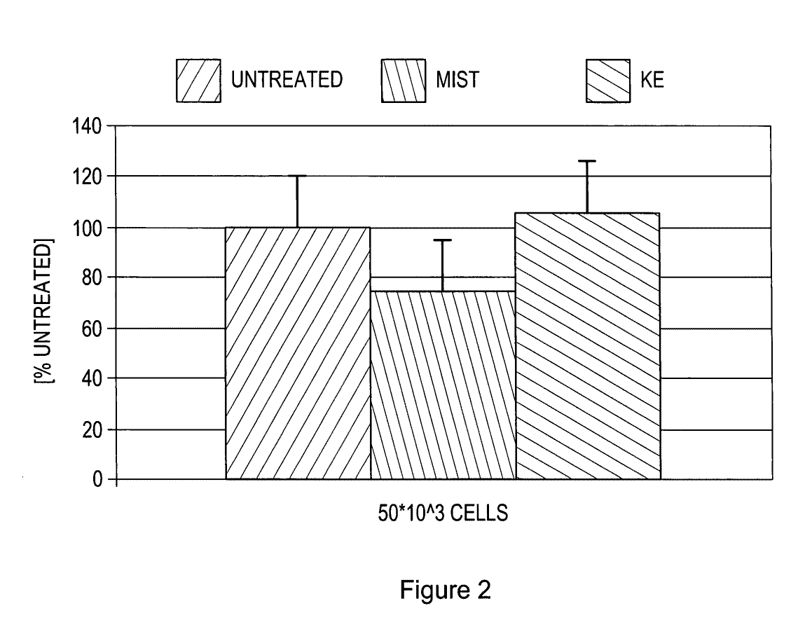

FIG. 2 shows that low frequency ultrasound energy inhibits TNFα production in LPS stimulated cells.

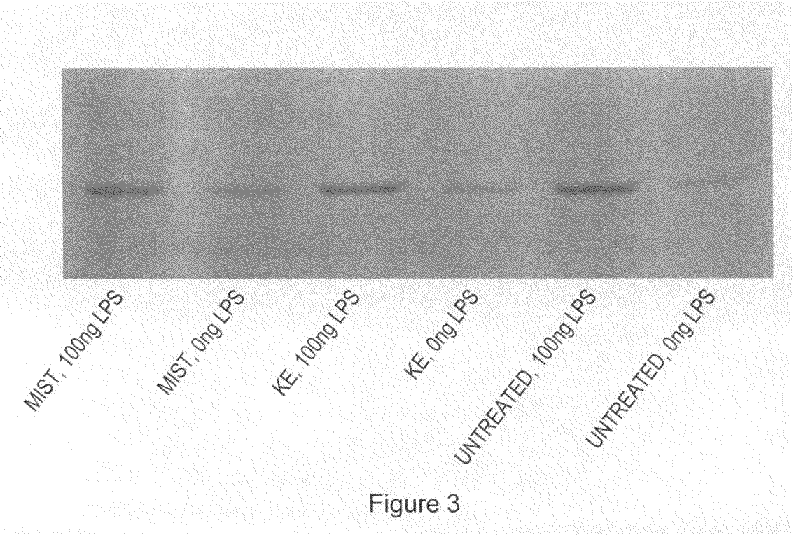

FIG. 3 shows that low frequency ultrasound energy inhibits p38 activation in LPS stimulated cells.

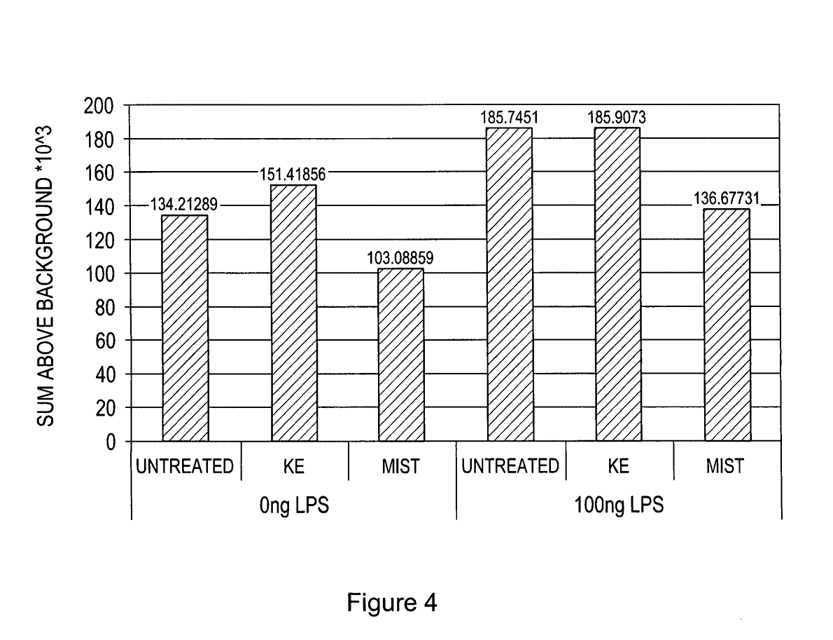

FIG. 4 shows that low frequency ultrasound energy inhibits p38 activation in LPS stimulated cells.

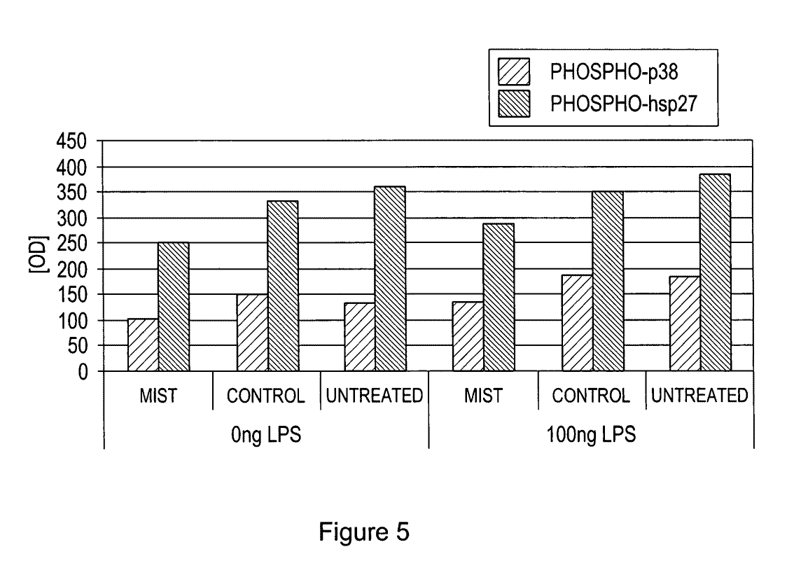

FIG. 5 shows that low frequency ultrasound energy inhibits hsp27 activation.

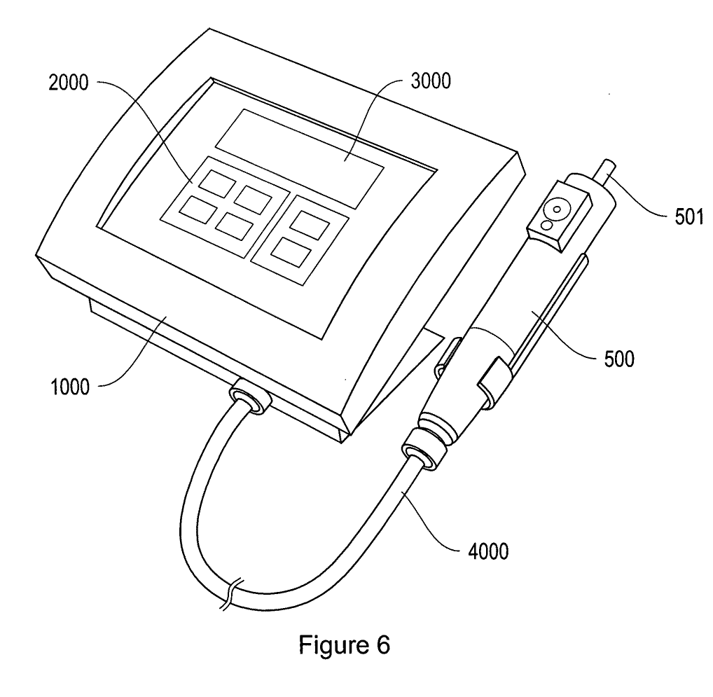

FIG. 6 shows an exemplary system for delivering ultrasonic energy to a patient.

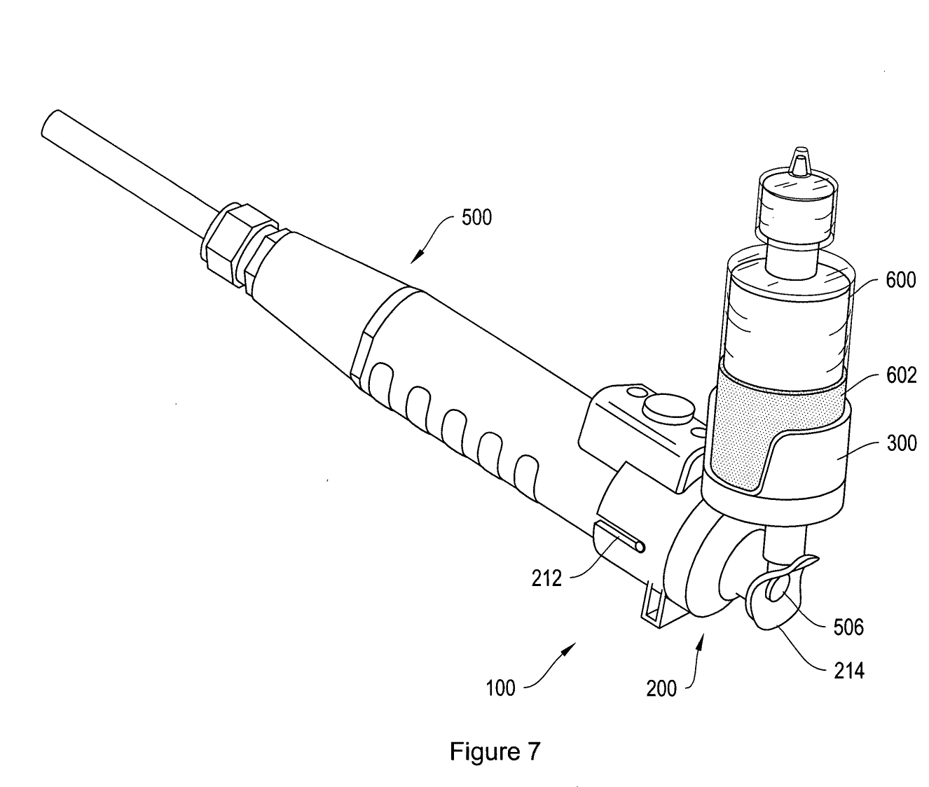

FIG. 7 shows an exemplary ultrasound transducer for delivering ultrasonic energy to a patient. The figure depicts an exemplary transducer, an applicator nozzle, and a fluid source.

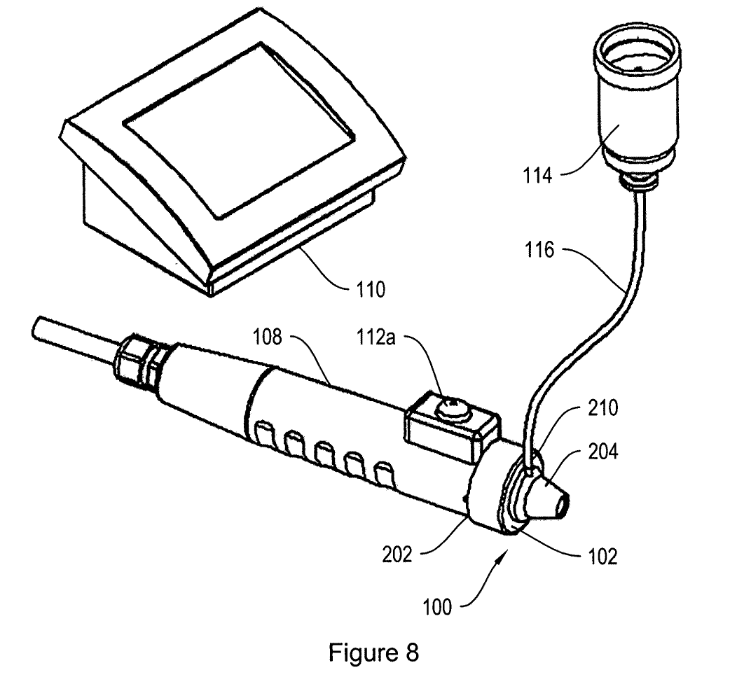

FIG. 8 shows another exemplary system for delivering ultrasonic energy to a patient. The figure depicts a system, which includes drive electronics and software for operating the device and providing information to the operator via a graphical user interface; an ultrasonic transducer; an applicator nozzle; and a fluid source.

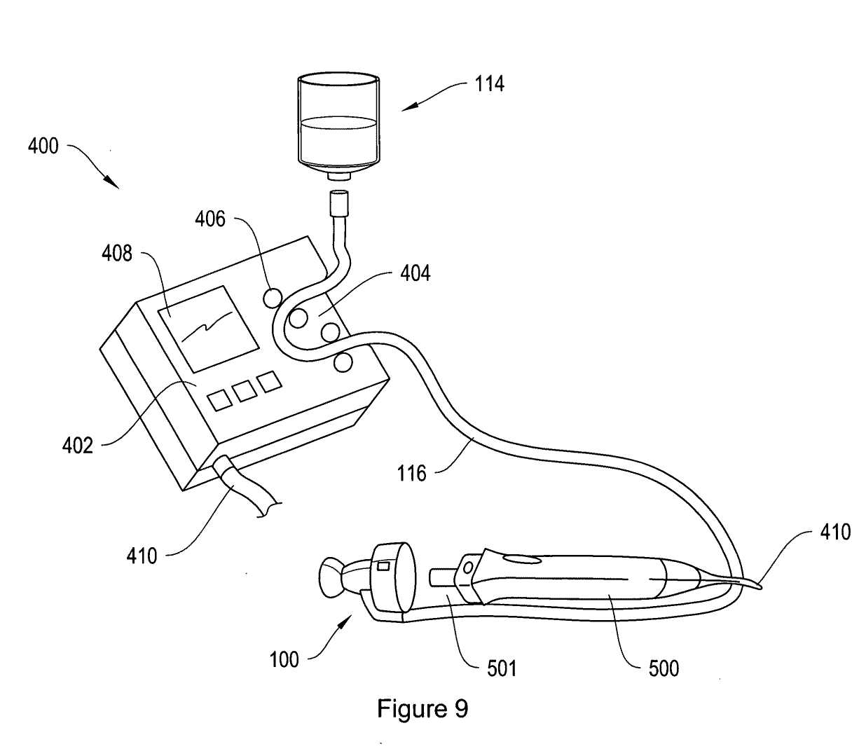

FIG. 9 shows another exemplary system for delivering ultrasonic energy to a patient. The figure depicts a system, which includes drive electronics and software for operating the device, controlling fluid flow, and providing information to the operator via a graphical user interface; an ultrasonic transducer; an applicator nozzle; and a fluid source.

### DETAILED DESCRIPTION OF THE INVENTION

Low frequency, non-contact ultrasound has been used in the treatment of wounds. U.S. Pat. No. 6,569,099, hereby incorporated by reference in its entirety, describes the use of ultrasound in wound therapy. Co-pending U.S. application Ser. Nos. 11/473,934, 60/878,621, and 12/006,739 describe particular transducer and applicator designs, and provide further description for using non-contact ultrasound in the treatment of wounds. Co-pending application Ser. No. 11/473,934, filed Jun. 23, 2006, is hereby incorporated by reference in its entirety. Co-pending application Ser. No. 60/878,621, filed Jan. 4, 2007, and co-pending application Ser. No. 12/006,739, filed Jan. 4, 2008, are hereby incorporated by reference in their entirety. The systems and devices provided in these co-pending applications are exemplary of the systems and devices that can be used to deliver ultrasonic energy to patient tissue to treat a rheumatoid disorder.

The present invention provides for the use of low frequency ultrasound, delivered at a non-contact distance, to treat or ameliorate symptoms of an inflammatory disorder. Specifically, the invention provides methods to treat or ameliorate the symptoms of a rheumatoid disorder. The methods described herein can be performed using, for example, the ultrasound therapy devices and systems disclosed in the above referenced co-pending applications. However, additional device configurations more specifically adapted for use in treating particular inflammatory disorders are also contemplated. Regardless of the specific device used, the invention provides methods for treating and ameliorating the symptoms of an inflammatory disorder by delivering low frequency ultrasound energy to effected tissue of a patient in need thereof. The low frequency ultrasound is delivered from a non-contact distance and without causing a substantial increase in the temperature of the treated tissue. In other words, the ultrasound energy is delivered to the effected tissue of the patient in need thereof, without contact between the ultrasound transducer, or other components of the device, and the effected tissue.

For the treatment of certain conditions, it may be preferable to have treatment conducted in a hospital or doctor's office so that a health care professional can monitor the duration and course of the treatment. Under certain circumstances, however, it may be preferable to allow the patient to be treated at home—either by a visiting health professional or by the patient himself.

The methods of the present invention can be used to treat or ameliorate one or more symptoms of an inflammatory disorder. In particular, the methods of the present invention can be used to treat or ameliorate one or more symptoms of a rheumatoid disorder. Rheumatoid disorders, as used herein, refer to any of a variety of inflammatory disorders characterized by inflammation, and sometimes degeneration and/or metabolic derangement, of the connective tissue structures, especially the joints and related structures. Rheumatoid disorders typically cause pain, stiffness, and/or limitation of motion

The inflammatory response is an important component of the immune system. However, the inflammatory response can destroy healthy tissue and cause tissue damage. In the case of rheumatoid disorders, patients may experience short term or long term symptoms including swelling, redness, fever, a rash or hives, pain, stiffness, and decreased range of motion. Depending on the duration and severity of the symptoms, as well as the effected joints or tissues, rheumatoid disorders can range from merely annoying to uncomfortable to disabling. Exemplary rheumatoid disorders include, but are not limited to, rheumatoid arthritis, juvenile arthritis, bursitis, spondylitis, gout, scleroderma, Still's disease, and vasculitis.

The present invention provides a method of treating an inflammatory disorder in a patient in need thereof, comprising delivering low frequency ultrasound energy from a non-contact distance to effected tissue of the patient in need thereof, wherein said low frequency ultrasound energy penetrates the tissue to provide a therapeutic effect to decrease symptoms of the inflammatory disorder. In certain embodiments, the inflammatory disorder is a rheumatoid disorder and the effected tissue is one or more joints. For such embodiments, the method comprises delivering ultrasound energy from a non-contact distance from the skin overlying the effected joint. The ultrasound energy penetrates the skin, thereby delivering a therapeutic effect to the underlying joint tissue. In certain embodiments, the inflammatory disorder is a rheumatoid disorder and the effected tissue is one or more blood vessels. For such embodiments, the method comprises delivering ultrasound energy from a non-contact distance from the skin overlying the effected vessels. The ultrasound energy penetrates the skin, thereby delivering a therapeutic effect to the underlying vessel tissue.

By “treating” is meant to include decreasing or eliminating one or more symptoms of the inflammatory disorder. Low frequency ultrasound energy is administered (with or without a liquid spray) to effected tissue of the patient. Specifically, low frequency ultrasound is delivered at a non-contact distance from the skin overlying effected connective tissue (e.g., joint, ligament, tendon, blood vessels, etc.). The low frequency ultrasound energy is administered without contact between the effected tissue (or the skin overlying the effected tissue) and the ultrasound transducer or other components of the device (non-contact distance). The low frequency ultrasound energy penetrates the tissue to provide a therapeutic effect. Without being bound by theory, over the course of one or more treatments, the ultrasound energy helps decrease the local inflammatory response, thus decreasing or eliminating the presence, severity, and/or frequency of the symptoms of the inflammatory disorder. Additionally, when the symptoms of the inflammatory disorder also include a rash or other skin manifestation, such skin-related symptoms are also treated by delivery of ultrasound in this manner. Regardless of the mechanism of action of the ultrasound energy, these methods can be effectively used to treat patients.

In certain embodiments, the inflammatory disorder is a rheumatoid disorder. Rheumatoid disorders, as used herein, refer to any of a variety of inflammatory disorders characterized by inflammation, and sometimes degeneration and/or metabolic derangement, of the connective tissue structures, especially the joints, ligaments, and tendons. Rheumatoid disorders typically result in pain, stiffness, and/or limitation of motion. The particular tissue or tissues effected depends on the rheumatoid disorder. Exemplary rheumatoid disorders include, but are not limited to, rheumatoid arthritis, juvenile arthritis, bursitis, spondylitis, gout, scleroderma, Still's disease, and vasculitis.

In certain embodiments, the rheumatoid disorder is rheumatoid arthritis and “treating” rheumatoid arthritis includes decreasing the severity, frequency, and/or occurrence of one or more of the symptoms of rheumatoid arthritis. In other embodiments, the rheumatoid disorder is juvenile arthritis and “treating” includes decreasing the severity, frequency, and/or occurrence of one or more of the symptoms of juvenile arthritis. In other embodiments, the rheumatoid disorder is bursitis and “treating” includes decreasing the severity, frequency, and/or occurrence of any one or more of the symptoms of bursitis. In other embodiments, the rheumatoid disorder is spondylitis and “treating” includes decreasing the severity, frequency, and/or occurrence of any one or more of the symptoms of spondylitis. In other embodiments, the rheumatoid disorder is gout and “treating” includes decreasing the severity, frequency, and/or occurrence of any one or more of the symptoms of gout. In other embodiments, the rheumatoid disorder is scleroderma and “treating” includes decreasing the severity, frequency, and/or occurrence of any one or more of the symptoms of scleroderma. In other embodiments, the rheumatoid disorder is Still's disease and “treating” includes decreasing the severity, frequency, and/or occurrence of any one or more of the symptoms of Still's disease. In other embodiments, the rheumatoid disorder is vasculitis and “treating” includes decreasing the severity, frequency, and/or occurrence of any one or more of the symptoms of vasculitis.

Exemplary symptoms include, but are not limited to, redness, swelling, inflammation, fever, decreased range of motion, and pain. Examples of reducing the occurrence or severity of symptoms include, but are not limited to, decreasing the number of swollen joints, decreasing the number of painful joints, decreasing the reliance on pain medication, decreasing a patient's self-evaluation of the frequency or severity of their pain, increasing freedom of motion, increasing mobility, decreasing fever, and increasing the ability to perform daily tasks.

Low frequency ultrasound energy can be delivered alone. Such methods are often referred to as delivering low frequency ultrasound “dry”. In other words, in certain embodiments, the treatment method comprises delivering low frequency ultrasound (from a non-contact distance) in the absence of a liquid spray or other coupling agent. When used in this way, the ultrasound energy penetrates the tissue to provide a therapeutic effect. Over one or more treatments, improvement in the patient's condition is observed.

Alternatively, the low frequency ultrasound energy can be delivered via a liquid spray. Such methods are often referred to as delivering low frequency ultrasound “wet”. In other words, a combination of ultrasound energy and a liquid spray is delivered (from a non-contact distance) to the tissue. The energy, and to some extent the liquid spray, penetrate the tissue to provide a therapeutic effect. Exemplary liquids that can be used to generate a liquid spray include saline or water. Alternatively, the liquids used to generate the spray can themselves be (or contain) a therapeutic agent, such as an antibiotic, anti-inflammatory, steroid, analgesic, antiseptic, and the like.

In certain embodiments, the method comprises very local delivery of ultrasound energy (in the presence or absence of a liquid spray) to the effected tissue. In other words, the goal is to treat, to the extent possible, only effected tissue and not asymptomatic tissue. In other embodiments, the method comprises local delivery that includes effected tissue, as well as adjacent tissue—even if such adjacent tissue is asymptomatic. In other embodiments, the method comprises treating the entire region. For example, if a patient has arthritis symptoms in the joints of the left index finder and thumb, low frequency ultrasound would be delivered, for example, to the entire left hand and left wrist, as well as the right hand and right wrist. Under such circumstances, ultrasound energy could additionally be delivered to other joints, including the joints of the toes, feet, knees, ankles, and elbow. The patient's health professional can select the appropriate treatment approach, including the number of treatments, the duration of each treatment, and whether the treatment should be “dry” or “wet”.

In certain embodiments, the method for treating an inflammatory disorder comprises multiple treatments. For example, patients may receive doses of ultrasound two or more times per week, for one, two, three, four, or more than four weeks. The appropriate number of treatments, and the duration of each treatment, can be determined by a health care provider based on, for example, the particular inflammatory disorder being treated, the severity of the disorder, and the overall health of the patient. In certain embodiments, each treatment comprises delivering ultrasonic energy to patient tissue for at least approximately one minute or for at least approximately two consecutive minutes. In certain embodiments, each treatment comprises delivering ultrasonic energy to patient tissue for at least approximately 3 minutes, at least approximately 4 minutes, or at least approximately 5 minutes. In certain embodiments, each treatment comprises delivering ultrasonic energy to patient tissue for at least approximately 6 minutes, at least approximately 7 minutes, at least approximately 8 minutes, at least approximately 9 minutes, or at least approximately 10 minutes. In other embodiments, each treatment comprises delivering ultrasonic energy for approximately 5-10 minutes, approximately 10-15, or approximately 15-20 minutes.

In certain embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., about 5, 6, 7, 8, 9, or even about 10 millimeters).

In certain embodiments, the therapeutic effect includes decreasing an inflammatory response, as assayed by expression of TNF-α or other inflammatory cytokine. Therapeutic efficacy also includes one or more of decreasing bacterial count, increasing healing, decreasing swelling, decreasing patient-reported pain, decreasing reliance on pain medication, improvement in mobility, improvement in range of motion, decrease in the number of swollen joints, decrease in the number of painful joints, and increase in the ability to perform daily task. Over the course of therapy, therapeutic efficacy can be assessed by evaluating improvement in the presence or severity of the symptoms of the inflammatory disorder.

In certain embodiments, the low frequency ultrasound energy delivered is approximately 10-100 kHz, approximately 20-80 kHz, approximately 20-40 kHz, approximately 35-60 kHz, or approximately, 40-50 kHz.

In certain embodiments, the low frequency ultrasound energy is also low intensity ultrasound energy. Intensity refers to the amount of energy transferred to the tissue. In certain embodiments, the low frequency, low intensity energy has an intensity of approximately 0.1 to 2.2 W/cm². In certain embodiments, the low frequency, low intensity energy has an intensity of approximately 0.1 to 0.75 W/cm² In certain embodiments, the low frequency, low intensity energy has an intensity of approximately 0.4-0.7 W/cm². In certain embodiments, the low frequency, low intensity energy has an intensity of approximately 0.5 W/cm².

In certain embodiments, separation distance (non-contact distance) between the distal most surface of the ultrasound therapy device and the skin overlying the patient tissue being treated is a non-contact distance of at least 0.1 inches (2.5 mm). Preferably, the separation distance is from about 2.5 mm to about 51 cm, more preferably, from about 15 mm to about 25 mm. Regardless of the exact distance, non-contact treatment means that there is no contact between the ultrasound device and the skin overlying the effected tissue that is being treated. However, in certain embodiments, it is possible that components of the device may contact patient tissue that is not being subjected to treatment. For example, to facilitate delivery of the ultrasound energy, a handle of the device may be affixed to the patient's arm, thereby alleviating the need for an operator to hold the device throughout treatment. Such contact with other patient tissue that is not being subjected to treatment does not alter the characterization of the treatment as “non-contact”.

In certain embodiments, the low frequency ultrasound does not result in a significant increase in the temperature of the skin overlying the tissue being treated.

In certain embodiments, the low frequency ultrasound decreases the symptoms of the rheumatoid disorder and also reduces skin-related manifestations of the rheumatoid disorder.

In certain embodiments, the low frequency ultrasound energy does not significantly decrease the viability of human cells of the effected tissue or overlying skin.

In certain embodiments, low frequency ultrasound therapy is part of a therapeutic regimen used in conjunction with one or more additional treatment modalities. For example, a patient may also receive topical or oral medications, or local or systemic injections. Additionally, patients can use diet, acupuncture, stress management, exercise, occupational therapy, physical therapy, or other herbal or homeopathic therapies to help manage the symptoms of their inflammatory disorder.

In certain embodiments, the effected tissue is selected from one or more of joints, tendons, ligaments, or blood vessels. In certain embodiments, the effected tissue is one or more joints. In certain embodiments, the effected joints are one or more of fingers, toes, wrists, ankles, elbows, knees, and hips. Without being bound by theory, joints and other connective tissue are often relatively close to the overlying skin. Accordingly, ultrasound energy readily penetrates the skin and provides a therapeutic effect to the underlying connective tissue.

In another aspect, the invention provides a method for reducing pain associated with a rheumatoid disorder in a patient in need thereof. The method comprises providing a transducer which can emit low frequency ultrasonic energy. Ultrasonic energy is delivered to effected tissue of said patient. The energy is delivered from a non-contact distance between a vibrating tip of the transducer and the effected tissue of said patient. The delivered ultrasonic energy provides a therapeutic effect to reduce pain associated with the rheumatoid disorder.

In certain embodiments, the effected tissue treated is selected from one or more of joints, tendons, and ligaments.

In certain embodiments, the ultrasonic energy is delivered via a liquid spray, and the method comprises delivering the low frequency ultrasonic energy and the liquid spray to said patient. In other embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the effected tissue or administering a systemic medicament prior to and/or following delivering said ultrasonic energy. In certain embodiments, the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

In certain embodiments, the step of delivering ultrasonic energy comprises delivering ultrasonic energy for at least about 2 consecutive minutes (e.g., this defines one treatment). In certain embodiments, the method comprises delivering ultrasonic energy at least twice per week (e.g., at least two treatments per week) for at least two weeks.

In certain embodiments, the rheumatoid disorder is rheumatoid arthritis or juvenile arthritis. In certain embodiments, the rheumatoid disorder is selected from any of bursitis, spondylitis, gout, scleroderma, Still's disease, and vasculitis.

In another aspect, the invention provides a method for treating a rheumatoid disorder. The method comprises providing a transducer which can emit low frequency ultrasonic energy and delivering said ultrasonic energy to a patient in need thereof. The ultrasonic energy is delivered from a non-contact distance between a vibrating tip of the transducer and treated patient tissue, and the delivered ultrasonic energy provides a therapeutic effect to decrease one or more symptoms of the rheumatoid disorder in said patient.

In certain embodiments, the ultrasonic energy is delivered to at least one of said patient's joints, ligaments, or tendons.

In certain embodiments, the ultrasonic energy penetrates the patient tissue to provide a therapeutic effect.

In certain embodiments, the ultrasonic energy is delivered via a liquid spray, and the method comprises delivering the low frequency ultrasonic energy and the liquid spray to the patient. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the treated tissue or systemically administering medicament prior to and/or following delivering said ultrasonic energy. In certain embodiments, the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

In certain embodiments, delivering ultrasonic energy comprises delivering ultrasonic energy for at least about 2 consecutive minutes (e.g., this defines one treatment). In certain embodiments, the method comprises delivering ultrasonic energy at least twice per week for at least two weeks.

In certain embodiments, the one or more symptoms are selected from one or more of inflammation, pain, tingling, weakness, decreased grip strength, swelling, itchiness, burning, fever, and decreased range of motion.

In certain embodiments, the rheumatoid disorder is rheumatoid arthritis or juvenile arthritis. In other embodiments, the rheumatoid disorder is selected from any of bursitis, spondylitis, gout, scleroderma, Still's disease, and vasculitis.

In another aspect, the invention provides a method for managing symptoms of a rheumatoid disorder. The method comprises providing a transducer which can emit low frequency ultrasonic energy and delivering said ultrasonic energy to a patient in need thereof for at least two consecutive minutes at least twice per week (e.g., providing at least 2 treatments/week). The ultrasonic energy is delivered from a non-contact distance between a vibrating tip of the transducer and treated patient tissue, and the delivered ultrasonic energy provides a therapeutic effect to treat one or more symptoms of the rheumatoid disorder.

In another aspect, the invention provides a method for decreasing the number of painful or swollen joints in a patient suffering from a rheumatoid disorder. The method comprises providing a transducer which can emit low frequency ultrasonic energy and delivering said ultrasonic energy to said patient. The ultrasonic energy is delivered from a non-contact distance between a vibrating tip of the transducer and treated patient tissue, and the delivered ultrasonic energy provides a therapeutic effect to decrease the number of painful or swollen joints in said patient.

In another aspect, the invention provides a method for decreasing expression of an inflammatory cytokine in a patient having a rheumatoid disorder. The method comprises providing a transducer which can emit low frequency ultrasonic energy and delivering said ultrasonic energy to said patient in need thereof. The delivered ultrasonic energy decreases expression of said inflammatory cytokine in one or more of an effected joint, ligament, tendon, skin, or blood vessel of said patient, and the inflammatory cytokine is TNFα.

In certain embodiments, the ultrasonic energy is delivered via a liquid spray, and the method comprises delivering the low frequency ultrasonic energy and the liquid spray to the patient. In certain embodiments, the ultrasonic energy is delivered in the absence of a liquid spray or coupling agent.

In another aspect, the invention provides a method for decreasing and/or assessing expression of TNF-α in response to a stimulus. In certain embodiments, the method is an in vitro method used to modulate the expression of TNF-α in cells in vitro (e.g., in cells in culture). In certain embodiments, the method is an in vivo method (tissue is treated in the context of the patient or animal body) and efficacy is assessed by evaluating TNF-α expression following treatment in comparison to TNF-α treatment prior to or in the absence of treatment.

In certain embodiments, TNF-α expression is assessed in one or more cell types of the immune system, such as activated monocytes or macrophages. In certain embodiments, the stimulus is an allergen or irritant.

In certain embodiments, the method is used to assess the progress or effectiveness of the treatment of an inflammatory disease, such as a rheumatoid disorder.

In another aspect, the invention provides a method for modulating the expression of one or more inflammatory cytokines in response to a stimulus. In certain embodiments, the method is an in vivo method, wherein the tissue is treated in the context of the patient or animal, and cytokine expression is evaluated using either an in vitro or in vivo assay. In certain embodiments, the method is an in vitro method. In certain embodiments, the in vitro or in vivo method is used to modulate the expression of TNF-αt, the activation of p38, or the expression of one or more interleukin. Regardless of whether the energy is delivered to tissue in vivo or cells in vitro, cytokine expression can be evaluated using an assay performed within or outside the context of the organism (an in vivo or in vitro assay).

In certain embodiments, expression or activation of an inflammatory cytokine is assessed in one or more cell types of the immune system, such as activated monocytes or macrophages. In certain embodiments, the stimulus is an allergen or irritant.

In certain embodiments of any of the foregoing or following, the delivered ultrasonic energy decreases pain. Reduction in pain can be evaluated relative to the pain experienced, on average, by patients whose treatment does not include treatment with low frequency, non-contact ultrasonic energy. Additionally or alternatively, reduction in pain may be evaluated based on the amount and frequency of pain medication requested or required to sufficiently manage patient pain relative, on average, to that needed by patient's whose treatment does not include low frequency, non-contact ultrasonic energy therapy. Such methods for evaluating reduction in pain are merely exemplary. Any standard method used by physicians and health care providers to evaluate pain and pain management can also be utilized. A reduction in reliance on pain medication includes a reduction in the dosage of medication requested or required to control pain and/or a reduction in the frequency with which medication is requested or required to adequately control pain. A reduction in reliance on pain medication may also include a shift from narcotic-based pain medications to non-narcotic or other over the counter pain medication (for example, a shift from morphine to ibuprofen).

In certain embodiments, the low frequency ultrasonic energy delivered from a non-contact distance is non-thermal. In other words, delivery of the ultrasonic energy (and optionally liquid spray) does not cause a significant increase in the temperature of the treated patient tissue (e.g., does not increase the temperature of the treated patient tissue by more than approximately 1° F.).

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer, for example to a portion of the transducer tip.

In certain embodiments, the ultrasonic energy is delivered in the absence of a liquid spray or coupling agent. Delivery of ultrasonic energy in the absence of a liquid spray or coupling agent is sometimes referred to herein as “dry” delivery. As with “wet” delivery, the ultrasonic energy is delivered from a non-contact distance.

For any of the foregoing or following aspects and embodiments, the invention contemplates delivering low frequency ultrasonic energy. In certain embodiments, the ultrasonic energy is delivered at a frequency of approximately 200 kHz to 400 kHz. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

For any of the foregoing or following aspects and embodiments, the invention contemplates delivering low frequency ultrasonic energy so as to provide a certain energy level to patient tissue. In certain embodiments, the ultrasonic energy level provided to patient tissue is approximately 0.1-2.0 watts/cm². In certain embodiments, the ultrasonic energy level provided to patient tissue is approximately 1.0-2.0 watts/cm². In certain embodiments, the ultrasonic energy level provided to patient tissue is approximately 0.1-1.0 watts/cm². In certain other embodiments, the ultrasonic energy level provided to patient tissue is approximately 0.1-0.7 watts/cm². In certain other embodiments, the ultrasonic energy level provided to patient tissue is approximately 0.5-1.0 watts/cm².

In certain embodiments, the methods of the present invention are repeated so that ultrasonic energy is delivered at least about twice per week for at least about two weeks. In other embodiments, ultrasonic energy is delivered at least about twice per week for at least about 3, 4, or 5 weeks. In other embodiments, ultrasonic energy is delivered at least about three times per week for at least about 2, 3, 4, or 5 weeks. In still other embodiments, the method includes one or more daily treatments for a least about 1, 2, 3, 4, or 5 weeks. In still other embodiments, the method includes one or more treatments per week for greater than six weeks. When multiple treatments are administered, each treatment may be of the same duration or of differing durations.

In certain embodiments of the foregoing or following aspects and embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., about 5, 6, 7, 8, 9, or even about 10 millimeters). In other embodiments, the delivered ultrasonic energy penetrates treated patient tissue to a depth of at least about 4 millimeters, at least about 5 millimeters, at least about 6 millimeters, at least about 8 millimeters, at least about 9 millimeters, or at least about 10 millimeters. In other embodiments, the delivered ultrasonic energy penetrates treated patient tissue to a depth of greater than 10 millimeters. Without being bound by theory, one of skill in the art will appreciate that when all or a portion of the therapeutic benefit of ultrasonic energy is due to penetration of the energy below the tissue surface, that this indicates that an effective amount of energy penetrates to an effective depth. However, such an effective amount need not be and is likely not the same energy level as that which initially contacts the tissue surface.

Without being bound by theory, in certain embodiments, and regardless of whether and to what depth the emitted ultrasonic energy penetrates patient tissue, the emitted energy may provide a therapeutic effect at the tissue surface, and/or via a relay mechanism from the tissue surface to underlying tissue, and/or by penetrating treated tissue.

In certain embodiments of any of the foregoing, ultrasound energy is delivered from a non-contact distance “dry”. In certain embodiments of any of the foregoing, ultrasound energy is delivered from a non-contact distance in the presence of a liquid mist (“wet”). The liquid mist is generated by contacting, dripping, or otherwise delivering a liquid to a portion of a vibrating ultrasound transducer, for example, a portion of the transducer tip, to create a spray. The spray and the ultrasound energy are directed to the patient tissue. When ultrasound energy is delivered “wet”, the liquid may be an inert or substantially inert liquid such as saline solution, oil, Ringer's solution, sterile water, and the like. The liquid may also be or contain a therapeutic medicament including, but not limited to, a growth factor, antibiotic, antifungal, antimicrobial, analgesic, anti-inflammatory, hypochlorous acid, or angiogenesis promoting agent. In certain embodiments, the fluid spray produced has a substantially uniform particle size. Exemplary fluids include, but are not limited to, sterile water, oxygenated water, saline solution, oil, or other isotonic or hypertonic solution. In certain embodiments, the fluid does not contain a therapeutic drug (e.g., the fluid is substantially free from a drug). In certain other embodiments, the fluid further includes one or more therapeutic drugs such as antibiotics, anti-fungals, anti-virals, growth factors, analgesics, narcotics, and the like. When the fluid includes a therapeutic drug, the drug may be formulated in any of the foregoing fluids (e.g., water, saline, etc), or the drug may be formulated in another pharmaceutically acceptable carrier appropriate for the formulation of the particular drug. In certain embodiments, the fluid (whether including a therapeutic drug or free from therapeutic drug) further includes one or more preservatives appropriate for extending the shelf-life of the fluid. In one embodiment of any of the foregoing, the fluid (whether including a therapeutic drug or free from therapeutic drug) is sterile (e.g., the fluid is sterilized prior to or after it is added to the bottle).

The invention contemplates combinations of one or more of any of the foregoing or following aspects and embodiments of the invention.

### (ii) Definitions

Unless defined otherwise, all technical and scientific terms have the same meaning as is commonly understood by one of skill in the art to which this invention belongs.

The articles “a” and “an” are used herein to refer to one or to more than one (i.e., to at least one) of the grammatical object of the article. By way of example, “an element” means one element or more than one element.

By “treatment” is meant to refer to a session during which ultrasonic energy is delivered to patient tissue. Typically, a treatment is at least 1 consecutive minute in length.

The term “low frequency”, with respect to ultrasound energy, refers to frequencies less than approximately 500 kHz.

The term “non-contact” is used to refer to methods for delivering ultrasonic energy to patient tissue without direct contact between the treated patient tissue and the distal end of the ultrasound delivery device. When non-contact methods for delivering ultrasonic energy are used, the ultrasound transducer (including the transducer tip portion) does not contact (directly or via a coupling gel) the treated patient tissue. The non-contact distance can be measured as the distance between the distal most surface of the ultrasound transducer tip and the treated patient tissue or the non-contact distance can be measured as the distance between the distal most surface of an applicator nozzle and treated patient tissue. Exemplary non-contact distances are at least about 0.1 inches (2.5 mm) or from about 2.5 mm to about 51 cm or from about 15 mm to about 25 mm. However, recitation of an approximate non-contact distance does not indicate that the exact distance is maintained for the entire treatment time. More importantly, the term non-contact is used to indicate that there is no contact with the treated tissue. However, it is possible and permissible that components of the applicator or device may contact patient tissue that is not the intended target of treatment. For example, to facilitate delivery of the ultrasound energy, a handle of the device may be affixed to a patient's arm, thereby alleviating the need for an operator to hold the device throughout treatment. Such contact with other patient tissue that is not the intended target of treatment does not alter the characterization of the treatment as “non-contact”.

The term “applicator” and “applicator nozzle” are used interchangeably to refer to an optional portion of an ultrasound therapy device. When present, the nozzle shields the transducer tip and prevents inadvertent contact with the transducer tip when the device is in operation. Additionally, the applicator nozzle can be used as part of the mechanism for delivering fluid to a portion of the transducer and/or as part of the mechanism for directing the delivered ultrasonic energy and/or liquid spray to patient tissue. Exemplary applicator nozzles are depicted herein. However, other applicator nozzles, as well as transducer assembly designs that do not include an applicator nozzle can similarly be used.

The terms “ultrasonic energy” and “ultrasound energy” are used interchangeably herein.

### (iii) Systems, Devices and Methods for Delivering Ultrasonic Energy

Numerous systems and devices for delivering ultrasonic energy are available. Such existing devices, as well as modifications and improvements thereof, are exemplary of systems and devices that may be used to deliver low frequency ultrasonic energy to patient tissue. In certain embodiments, low frequency ultrasonic energy is delivered from a non-contact distance and without direct contact with treated patient tissue. For example, the low frequency ultrasonic energy (in the presence or absence of liquid spray) is delivered from a non-contact distance between the treated patient tissue and the transducer tip of the ultrasound device and/or the applicator nozzle.

FIG. 6 depicts an exemplary system for delivering ultrasonic energy. An exemplary ultrasound therapy device includes a transducer assembly 500 operatively connected via a connector 4000 to a generator 1000. As described herein, the ultrasound therapy device may further include an applicator 100 (not shown in FIG. 6) that can be interconnected to the transducer assembly 500, thereby shielding the transducer tip portion 501.

Briefly, the generator 1000 includes the components necessary to supply power to the transducer assembly 500, and also contains a control panel 2000, and a graphical user interface (GUI) 3000 for displaying information helpful to the operator. The generator 1000 includes three major functional sections: the AC MAINS, the main board, and the GUI board. The local AC MAINS is connected to an appliance inlet with a hospital grade detachable power cord. The appliance inlet is a power entry module listed for medical applications. In certain embodiments, the appliance inlet is a power entry module with an 115V/230V voltage selection, and is designed to operate on 115 Vac and 60 Hz (e.g., for operation in North America) or 230 Vac and 50 Hz (e.g., for operation in Europe).

The MAIN board converts the secondary output voltage from the MAINS transformer to the low voltage power rails for the internal electronics and the drive voltage for the drive electronics to the transducer assembly. The MAIN board contains a microprocessor that controls, measures, and monitors the drive electronics. The transducer assembly connects to the MAIN board. The microprocessor, referred to as the engine, monitors the performance of the system and communicates the information to a second microprocessor located on the GUI board. In certain embodiments, the engine communicates to the second microprocessor via a RS-232 communication link. In certain embodiments, the electronics drive the ultrasound portion of the drive electronics with a push-pull converter that has a feedback loop with a Phase Locked Loop (PLL) to track the center frequency of the ultrasound components.

The GUI board provides the graphical user interface for the operator. A custom membrane switch panel with, for example 6 keys, allows the operator to select the functions and operating parameters of the system. A purchased graphical LCD display, connected to the GUI board, can be used to display information to the operator. For example, information about the system's status, mode of operation, and treatment time can be displayed via the GUI. The GUI may have a back light generator for the LCD on it. The GUI microprocessor runs the system by controlling the human interface and running various algorithms to control the operation of the system. For example, a treatment algorithm can be run on the GUI microprocessor. In certain embodiments, the system may include one or more of a timer to record total treatment time, a timer to count-down from a selected treatment time to zero, and an alarm to indicate that the total treatment time has elapsed or that there is a problem with some component of the device.

FIG. 6 also depicts an example of a transducer assembly 500. As depicted, only the transducer tip portion 501 is visible. The remainder of the transducer is contained within the plastic casing of the assembly. As depicted, the transducer tip portion 501 is uncovered. In operation, the transducer tip portion 501 may be shielded with, for example, an applicator nozzle. Exemplary applicator nozzles 100 are depicted in FIGS. 7-9. When used, an applicator nozzle helps prevent inadvertent contact of either the operator or the patient with the vibrating tip portion of the transducer. Additionally, an applicator nozzle can be used as part of the mechanism for directing the delivered ultrasonic energy to patient tissue. When the system is used “wet”, the applicator nozzle may also be used to deliver fluid to the transducer tip portion and to direct the delivered ultrasonic energy and the fluid spray to patient tissue.

The system depicted in FIG. 6 is currently sold by Celleration, Inc. as part of the MIST Therapy® System.

FIG. 7 shows an example of a portion of a system for delivering ultrasonic energy. Specifically, FIG. 7 shows a transducer assembly 500 interconnected to one embodiment of an applicator nozzle 100. The transducer assembly can be operatively interconnected to a generator, for example generator 1000 shown in FIG. 6.

As depicted, the transducer assembly 500 and applicator nozzle 100 are ready to be used “wet”. Specifically, a fluid bottle 600 containing fluid 602 is interfitted to a portion of the applicator nozzle so that fluid can be delivered to the transducer tip portion, and so that ultrasonic energy and a fluid spray can be delivered to patient tissue. As depicted, a fluid bottle 600 is interfitted to a cup portion 300 of the applicator nozzle. As shown, fluid delivery to the transducer would largely be gravity driven. However, pressure delivery methods, peristaltic pumps, fluid cartridges affixed directly to or housed within the transducer assembly, and the like are similarly contemplated. An alternative mechanism for providing fluid to the transducer is via a sock, membrane, film, or other means to wick fluid from a fluid container or fluid line to all or a portion of the transducer.

In certain embodiments, an applicator, as described herein, is interconnected with an ultrasound therapy device and used to deliver ultrasound energy (in the presence or absence of a liquid spray) to patient tissue. When used in this manner, the ultrasound energy (and liquid spray, if present) is delivered without contact between the applicator and the patient tissue being treated. In other words, the ultrasound energy (and liquid spray, if present) are delivered from a non-contact distance. Once delivered, the ultrasound energy acts at the cell surface and/or penetrates the treated tissue to provide a therapeutic effect.

The transducer assembly 500 and applicator nozzle 100 depicted in FIG. 7 are currently sold by Celleration, Inc. as part of the MIST Therapy® System. As depicted, applicator 100 generally includes a nozzle 200 and a cup 300. However, applicator designs that exclude the cup 300 can be readily used.

When included in the applicator design, the cup 300 may be designed to hold at least a portion of a bottle 600 therein. The bottle 600 generally holds a fluid 602, which may be saline. The fluid may alternatively be sterile water or some other isotonic or hypertonic solution or combination of solutions. The fluid may consist entirely or essentially of the saline or other similar solution, or the fluid may optionally include a therapeutic drug. The fluid may optionally be sterilized.

The applicator 100 is mechanically connectable with a transducer assembly 500 of an ultrasound therapy device. When activated, the transducer assembly 500 produces ultrasonic waves having a frequency and capable of delivering ultrasonic energy to patient tissue.

The proximal portion of the nozzle 200 slides over a distal portion of the transducer assembly 500. The plurality of aligning slots 212 (illustrated as two slots) of the nozzle 200 engage with a plurality of aligning pins of the transducer assembly 500. When connected, the distal end 506 of a transducer tip portion of the transducer assembly 500 may extend distally of the distal opening 214 of the nozzle 200 but not to a location that is distal of the tip 205 of the nozzle 200.

The fluid 602 to be sprayed and provided within the bottle 600 (or other appropriate fluid container or vessel) can be any appropriate carrier, such as saline, water (regular or distilled), or oil to be applied to tissue, such as a vegetable, peanut, or canola oil, optionally with a soluble pharmaceutical (e.g., an antibiotic), antiseptic, conditioner, surfactant, emollient, or other active ingredient. The fluid 602 can also be a combination of two or more fluids and/or substances having microscopic particles, such as powder and the like. Exemplary fluids include, but are not limited to, sterile water, saline solution, oil, oxygenated water, or other isotonic or hypertonic solutions. Exemplary fluids may, in certain embodiments, further include drugs (e.g., therapeutic agents) such as antibiotics, anti-fungals, anti-virals, growth factors, analgesics, narcotics, and the like, formulated in any of the foregoing fluids or in other pharmaceutically acceptable fluids appropriate for the fonmulation of the particular drug. However, in certain embodiments, the fluid does not include a drug. The fluid may be sterilized so that, in use, a spray of a sterile solution is administered to patients.

It is envisioned for the bottle 600 of the present disclosure to be eliminated and/or replaced with another structure for delivering the fluid 602 to the transducer assembly 500, such as a fluid bag or integrated cartridge or canister (not shown). In such an embodiment, the fluid 602 may optionally be delivered to the transducer assembly 500 in a pressurized state. Desirably, the pressurized fluid 602 in such an embodiment may be approximately equal to the pressure of the fluid 602 exiting the bottle 600, as in the previous embodiment.

FIG. 8 shows another example of a portion of a system for delivering ultrasonic energy. Specifically, FIG. 8 shows a generator 1000, a transducer assembly 500, and an alternative design for an applicator nozzle 100.

FIG. 8 depicts an applicator 100. As depicted the applicator 100 is interconnected to a transducer assembly 500. The applicator 100 is also interconnected to a fluid source 114 via a flexible tubing 116.

FIG. 8 also shows a switch 112a that may control one or more of the power supplied to the transducer assembly 500, the flow of fluid, or the fluid flow rate. Also shown is a fluid source 114 and tubing 116 that interconnects the fluid source 114 to the applicator 100 via a connector 210. As depicted, the connector comprises an opening in communication with the interior of the applicator 100, thereby providing a conduit to deliver fluid to a portion of the transducer.

FIG. 9 shows another example of a portion of a system for delivering ultrasonic energy. Specifically, FIG. 4 shows a pump-generator 400, a transducer assembly 500, and an alternative design for an applicator nozzle 100.

As depicted, the transducer assembly and applicator are interconnected to a fluid source 114 via flexible tubing 116. The applicator 100 is depicted just prior to interconnection to the transducer assembly 500. The transducer tip portion 501 is visible. When present, and once the applicator 100 is interfitted to the transducer assembly 500, the transducer tip portion 501 will be shielded, thereby preventing inadvertent contact with the transducer tip portion 501.

In this depiction, the pump-generator 400 includes additional mechanisms for controlling fluid delivery to the transducer assembly 500, the transducer tip portion 501, and the applicator 100. The depicted system provides an example of a fluid delivery mechanism that is not gravity fed, but rather under direct control of the user. The use of a peristaltic pump, such as the pump depicted, permits additional control over the rate at which fluid is delivered to the transducer.

An exemplary peristaltic pump at least includes a rotor and rollers or other tube-engaging members movable within a housing relative to the clamped flexible tubing. A peristaltic pump typically includes between four to six rollers. The rollers compress the clamped flexible tubing. As the rotor turns, the part of the tube under compression gets pinched and the pinching motion forces the fluid to move through the tube. The rollers relax the clamped flexible tubing as the rotor turns and the flexible tubing opens to its original state to induce fluid flow. FIG. 9 shows a fluid container 114, a tubing 116, an applicator 100, and a generator-pump unit 400. The generator-pump unit 400 includes, among other things, a generator portion 402, a pump portion 404, multiple rollers 406, an LCD display 408, and a connection inlet 410. The generator portion 402 may automate the fluid to enter the nozzle by, for example, regulating a valve (not shown) coupled to the tubing 116. In addition, the pressure applied to the fluid may be automatically maintained by the generator 402 based on values supplied by the user from a user interface, such as a dial, coupled to the generator 402. In addition, the generator 402 may report to the user the monitored pressure readings in the LCD display 404 of the generator 402. Although not shown, the generator-pump unit 400 may include an outer cover to protect the rollers 406 and the flexible tubing. In certain embodiments, the generator-pump unit 400 is fully integrated such that it performs all of the functions of the generator 1000 depicted in FIG. 6.

Although not depicted in the foregoing figures, fluid flows into the nozzle and is delivered to a vibrating transducer tip portion 501. Fluid delivery can be, for example, gravity driven or mechanically or otherwise controlled. The fluid source can be separate from or integrated within the generator and/or transducer assembly. Fluid delivery can be along all or a portion of the transducer tip portion, including to a distal portion of the tip portion. Fluid is dripped, flowed, wicked, or otherwise applied to all or a portion of the transducer tip portion, including to a plurality of sections of the transducer tip portion. Ultimately, in operation, fluid is delivered from the distal radiation surface of the transducer tip portion and ultrasonic energy and a fluid spray is delivered to patient tissue. Fluid contacts the transducer tip portion and ultrasonic energy and a fluid spray are delivered from the distal end of the applicator nozzle.

FIGS. 6-9 are merely exemplary of systems and devices that can be used to deliver ultrasonic energy. Additionally, although not depicted, devices that are typically used to deliver ultrasonic energy via direct contact with patient tissue can be adapted for use at a non-contact distance as part of the instant methods. Similarly, although applicator nozzles are not required, when present, appropriate applicator nozzles include removable nozzles, disposable nozzles, and nozzles that are non-removable and/or non-disposable.

Regardless of whether the foregoing or functionally related or differing devices are used, and regardless of whether used “wet” or “dry”, ultrasonic energy is delivered to patient tissue without direct contact between the transducer tip and/or applicator nozzle and the treated patient tissue.

In certain embodiments, the generator includes a treatment algorithm that calculates an approximate treatment time. Alternatively, a physician or health professional can select the desired treatment time. For example, treatment time may be determined based on the area of the tissue for which treatment is desired.

Generally, treatment times vary from approximately 1 minute to approximately 30 minutes. However, shorter (approximately 45 seconds-1 minute) and longer (25-45 minutes) treatment times are contemplated. In certain embodiments, the length of time of a treatment comprises delivering ultrasonic energy to patient tissue for at least about 1 consecutive minute, at least about 2 minutes, at least about 3 minutes, or at least about 5 minutes. In certain embodiments, the length of time of a treatment comprises delivering ultrasonic energy to patient tissue for approximately 1-2 minutes, approximately 1-5 minutes, approximately 2-6 minutes, approximately 3-8 minutes, or approximately 4-10 minutes. In certain embodiments, the length of time of a treatment comprises delivering ultrasonic energy to patient tissue for approximately 5-15 minutes, approximately 5-20 minutes, or approximately 5-25 minutes. Note that the foregoing treatment times are approximate times per treatment. Thus, when the method comprises multiple treatments, the total treatment time over the course of one or more days, weeks, or months will be the aggregate of the individual treatment times.

In certain embodiments, the method comprises a single treatment (e.g., delivering ultrasonic energy once for a period of at least about 1 minute). In certain embodiments, the method comprises multiple treatments delivered over the course of one or more days, weeks, and/or months. In certain embodiments, the method comprises at least two treatments per week for at least two weeks. Note that when the method comprises multiple treatments, each treatment may be of the same duration or of differing durations.

Once emitted energy, and fluid spray when applicable, emerge from the device, the operator directs the energy to the selected treatment site. In one embodiment, the treatment site is treated by slowly moving the applicator head back and forth and/or up and down (at a non-contact distance) across the site. The spray pattern may be, for example, serpentine or substantially checkerboard in pattern.

The above described delivery method (whether used “wet” or “dry”) has two advantages. First, this method helps insure that ultrasonic energy and liquid spray are delivered to the entire treatment site. Second, this method may help prevent operator fatigue that would likely result if the device was held in substantially the same place throughout the treatment. In one embodiment, the applicator is held such that the ultrasonic energy and liquid spray are delivered substantially normal to the surface of the treatment site. Additionally, the spray pattern may include moving the applicator in-and-out relative to the patient tissue (e.g., varying the distance from the patient while maintaining a non-contact distance). Such a spray pattern helps ensure that a treatment site is treated completely and at an effective distance. In other embodiments, the operator directs the energy to the treatment site by holding the applicator in substantially the same place throughout the treatment. Such a method is particularly useful when the treatment site is small and/or the treatment time is short. As noted above, the forgoing discussion of exemplary spray patterns are equally applicable whether ultrasonic energy is delivered “wet” or “dry”.

In one embodiment, the need for a human operator is eliminated. The transducer assembly is affixed to a robotic arm programmed to direct the emitted energy and liquid spray (when applicable) to the treatment site. The robotic arm can be programmed to hold the applicator in substantially the same place throughout treatment or to move the applicator (back and forth or in and out) relative to the treatment site.

As outlined above, in certain embodiments the emitted ultrasonic energy and fluid spray (when applicable) are applied to the treatment site for a treatment time proportional to the size of the treatment site. In one embodiment, the invention provides a treatment algorithm for selecting treatment time based on the size of the treatment site. For example, the time for each treatment is selected based on the area of the treatment site.

The present invention provides methods for using ultrasonic energy to treat rheumatoid disorders. By way of further non-limiting example, commonly-owned U.S. Pat. No. 6,569,099, and application Ser. Nos. 11/473,934, 10/409,272, 10/815,384, and 12/006,739 disclose ultrasonic systems and devices that can be used in the subject methods. The entire contents of each of the foregoing patents and patent application are incorporated herein by reference. Briefly, these patents and applications discloses devices, systems, and methods for delivering ultrasound energy, in the presence or absence of a liquid spray. The ultrasound energy and, when present the liquid spray, is delivered from a non-contact distance. Commonly-owned U.S. patent application Ser. Nos. 11/473,934 and 12/006,739, the entire contents of which are incorporated herein by reference, additionally provide several examples of removable applicator nozzles that can be used with an ultrasound therapy device. The disclosed devices and systems are exemplary of those that can be used to deliver ultrasonic energy to treat a rheumatoid disorder.

U.S. application Ser. Nos. 11/473,934, filed Jun. 23, 2006, 60/878,621, filed Jan. 4, 2007, and 12/006,739, filed Jan. 4, 2008, provide a detailed description of an ultrasound transducer and various applicator nozzle designs that can be used to deliver ultrasound energy (in the presence of absence of a liquid spray) to patient tissue from a non-contact distance. These applicator designs are exemplary of nozzle designs that can be used to deliver ultrasound energy as part of a treatment for an inflammatory disorder. The invention contemplates suitable combinations of any of the aspects and embodiments disclosed herein with the aspects and embodiments disclosed in application Ser. Nos. 11/473,934, 60/878,621, and 12/006,739, which are incorporated by reference in their entirety.

### (iv) Inflammatory Disorders

The present invention is based on the observation that low frequency ultrasound energy delivered from a non-contact distance decreases the inflammatory response. As such, the present invention can be used to decrease the inflammatory response, thus treating or ameliorating one or more symptoms of an inflammatory disorder. In particular, the present invention can be used to treat or ameliorate one or more symptoms of a rheumatoid disorder. Rheumatoid disorders, as used herein, refer to any of a variety of inflammatory disorders characterized by inflammation, and sometimes degeneration and/or metabolic derangement, of connective tissue structures. Exemplary connective tissues that can be affected include joints, ligaments, tendons, and blood vessels. Rheumatoid disorders are typically accompanied by pain, stiffness, and limitation of motion, and exemplary symptoms include pain, swelling, fever, lethargy, stiffness, and limitation of motion. Exemplary rheumatoid disorders include, but are not limited to, rheumatoid arthritis, juvenile arthritis, bursitis, spondylitis, gout, scleroderma, Still's disease, and vasculitis.

### Rheumatoid Arthritis

Rheumatoid arthritis is a long-lasting disease that can affect joints in any part of the body but most commonly the hands, wrists, and knees. With rheumatoid arthritis, the immune system mistakenly attacks itself and causes the joint lining to swell. The inflammation then spreads to the surrounding tissues, and can eventually damage cartilage and bone. In more severe cases, rheumatoid arthritis can affect other areas of the body, such as the skin, eyes, and nerves.

Symptoms of rheumatoid arthritis include, but are not limited to, fatigue, fever, rash, joint inflammation, pain, tenderness around the effected joints, stiffness, redness and warmth around the effected joints, and reduced range of motion.

Rheumatoid arthritis can occur at any age, but is commonly observed between the ages of 25 and 55. It is 2-3 times more common in women than in men. It is the second most common form of arthritis, affecting 2.1 million people in the U.S. alone.

Rheumatoid arthritis in some people may eventually cause the hands and feet to become misshapen as muscles weaken, tendons shrink, and the ends of bones become damaged. Current therapies include medications intended to decrease pain, joint swelling, and inflammation. These medications include non-steroidal anti-inflammatory medicines, corticosteroids, anti-mitotics (methotrexate and cyclophosphamides), and anti-TNFα medications intended to systemically dampen the inflammatory response. Additional treatments include diet, exercise, and physical therapy intended to help maintain muscle strength and range of motion, thereby slowing the disabling effects of the disease.

The methods of the present invention can be used in the treatment of rheumatoid arthritis. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating rheumatoid arthritis in a patient in need thereof. By “treating” is meant to include decreasing or eliminating one or more symptoms of rheumatoid arthritis. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of rheumatoid arthritis by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

### Juvenile Arthritis

Juvenile rheumatoid arthritis is characterized by chronic fever, anemia, and persistent arthritis in one or more joints that lasts at least six weeks. The disease can also have secondary effects on the heart, lungs, eyes, and nervous system.

Treatment is essentially the same as for adult rheumatoid arthritis, with heavy emphasis on physical therapy and exercise to help prevent disability as the body continues to grow. Rheumatoid arthritis is, at least in part, an autoimmune disorder that attacks cartilage.

Although treatments are available, many of the treatments involve the use of agents that may be particularly harmful when administered to children. As such, the development of alternative approaches is especially useful. The present invention provides such alternative treatment approaches.

The methods of the present invention can be used in the treatment of juvenile arthritis. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating juvenile arthritis in a patient in need thereof. By “treating” is meant to include decreasing or eliminating symptoms of juvenile arthritis. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of juvenile arthritis by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

### Bursitis

Bursitis is inflammation or irritation of the bursa. The bursa is a sac filled with lubricating fluid, located between tissues such as bone, muscle, tendons, and skin. The bursa acts to decrease rubbing, friction, and irritation between tissues.

Bursitis can be caused by repetitive strain or stress, or it may be caused by additional stress placed on the bursa due to another condition. For example, infection or inflammation due to gout, rheumatoid arthritis, or osteoarthritis can put stress on the bursa, thus leading to bursitis.

Bursitis typically effects the thumbs, elbows, shoulders, hips, knees, and achilles tendon. A patient may have symptoms effecting numerous tissue, or have symptoms at a single site. The extent of the bursitis is typically due to the underlying cause of the irritation of the bursa.

The most common symptom of bursitis is pain at the site of the bursa. The pain may also radiate out from the bursa. Loss of motion around the effected tissue is also a symptom of bursitis. For example, if the bursa around the shoulder is irritated, the patient may experience loss of motion or decreased range of motion in the shoulder. This is sometimes referred to as “adhesive capsulitis”.

Current treatment for bursitis focuses on avoiding activities that may exacerbate symptoms, rest, and/or the use of aspirin or other over-the-counter medications. The present invention provides an alternative therapy that can be used alone or in combination with other treatment modalities.

The methods of the present invention can be used in the treatment of bursitis. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating bursitis in a patient in need thereof. By “treating” is meant to include decreasing or eliminating symptoms of bursitis. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of bursitis by, for example, decrease the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

### Spondylitis

Spondylitis, also referred to as ankylosing spondylitis, refers to family of diseases that are a form of inflammatory arthritis. Spondylitis primarily affects the spine, although other joints and organs can become involved. Spondylitis, unlike many other rheumatic conditions is most common in young adults under 35. Some studies estimate that spondylitis occurs in approximately 1 in every 200 adults, making it as common as rheumatoid arthritis.

Ankylosing spondylitis causes inflammation of the spinal joints (vertebrae). Symptoms include severe, chronic pain; discomfort; and loss of range of motion. In the most advanced cases, the inflammation can lead to new bone formation on the spine, causing the spine to fuse. Once the spine is immobile, patients may become forced into unnatural postures. The hallmark feature of ankylosing spondylitis is the involvement of the sacroiliac (SI) joints at the base of the spine, where the spine joins the pelvis.

Although the spine is the primary affected tissue, spondylitis can also cause inflammation, pain and stiffness in other areas of the body, such as the shoulders, hips, ribs, heels and small joints of the hands and feet.

Current treatments are similar to those available for rheumatoid arthritis. Like rheumatoid arthritis, there is no known cure. The severity of the disease varies greatly from person to person. Some experience only intermittent back pain and discomfort, but others experience severe, and even disabling pain and loss of mobility.

The methods of the present invention can be used in the treatment of spondylitis. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating spondylitis in a patient in need thereof. By “treating” is meant to include decreasing or eliminating one or more symptoms of spondylitis. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of spondylitis by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

### Gout

Gout is a disease that causes sudden, severe episodes of pain and tenderness, redness, warmth, and inflammation in some joints. Gout is a dramatic example of a type of arthritis called “crystal arthritis,” sometimes called “microcrystalline arthritis” because the crystals that accumulate in the joints are very small. The crystals form in the joint space. As the body tries to remove the crystals, a painful inflammation occurs.

Gout usually affects one joint at a time, typically the large joint of the big toe. It also can affect other joints, including the knee, ankle, foot, hand, wrist, and elbow. In rare cases, it may affect the shoulders, hips, or spine.

Attacks of gout usually develop quickly, with the first symptoms typically occurring at night. During an episode of gout, patients often experience the following symptoms: sudden, severe joint pain; joint swelling; shiny red or purple skin around the joint; and extreme tenderness in the joint area.

At first, gout episodes are infrequent and last only about a week. Over time, however, attacks tend to occur more frequently and be of longer duration. Repeated episodes can damage the joint(s) leading to longer term joint stiffening and limited motion.

The methods of the present invention can be used in the treatment of gout. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating gout in a patient in need thereof. By “treating” is meant to include decreasing or eliminating symptoms of gout. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of gout by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

### Vasculitis

Vasculitis refers to inflammation of blood vessels. Blood vessels include the huge network of arteries and veins that deliver blood from the heart to all of the organs and tissues throughout the body, and then return the blood back to the heart.

Vasculitis can affect any blood vessel, although it seldom affects large veins. Further, depending on the type and extent of the disease, vasculitis can affect multiple blood vessels. Inflammation can affect the lining of the vessels (endothelium) or the wall of an artery or vein. This can cause the vessel to become thickened, weakened, narrowed, or scarred. The damaged vessel may not function normally, which can alter blood flow. Damaged blood vessels can lead to decreased blood flow, partial or complete organ failure due to lack of blood flow, or bleeding into the skin or other part of the body due to rupture of the blood vessel wall.

In some cases, vasculitis occurs in conjunction with another illness, such as lupus erythematosus or rheumatoid arthritis. In other cases, vasculitis is part of the body's reaction to a drug or other substance (so called, hypersensitivity vasculitis). In other cases, vasculitis occurs following or in conjunction with a viral infection, such as hepatitis B, hepatitis C, HIV, cytomegalovirus, Epstein Barr virus, or Parvo virus.

Vasculitis is typically classified according to the type and location of the effected blood vessels. For example, large vessel vasculitis affects large arteries. Medium sized vessel vasculitis affects medium sized arteries. Polyarteritis nodosa causes inflammation of medium to small arteries. Small vessel vasculitis affects small vessels (eg, very small arteries, arterioles, capillaries, and venules). Small and medium vessel vasculitis, sometime accompanies other inflammatory conditions (e.g., rheumatoid arthritis, systemic lupus erythematosus, inflammatory muscle diseases, and Sjögren's syndrome).

The optimal treatment depends upon the type of organ system involved and the severity of the condition. Symptoms of vasculitis vary from one patient to another and depend upon the type of vasculitis. Some common symptoms include: fatigue; weakness; fever; muscle and joint pain; lack of appetite and weight loss; abdominal pain; kidney symptoms (bloody urine, dark urine); and nerve symptoms (numbness, weakness, pain).

The methods of the present invention can be used in the treatment of vasculitis. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating vasculitis in a patient in need thereof. By “treating” is meant to include decreasing or eliminating one or more symptoms of vasculitis. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of vasculitis by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in swelling, decrease in pain, increased range of motion, increased ability to perform daily tasks, decreased reliance on pain or other medication, improvement in patient self-assessment of pain, quality of life, or other indicia.

### Scleroderma

Scleroderma is really a symptom of a group of diseases that involve the abnormal growth of connective tissue. It is thus often used as an umbrella term for these disorders. In some forms of scleroderma, hard, tight skin is the primary symptom, and other connective tissue is not significantly effected. In other forms of the disease, other connective tissues such as the blood vessels, joints, and muscles are effected. Sometimes, even the internal organs are effected by the disease.

Localized scleroderma is limited to the skin and related tissues and, in some cases, the muscle below. There are two generally recognized types of localized scleroderma. Morphea is characterized by local patches of scleroderma. The first signs of the disease are reddish patches of skin that thicken into firm, oval-shaped areas. The center of each patch becomes ivory colored with violet borders. These patches don't generally sweat or grow hair. Patches appear most often on the chest, stomach, and back, although they sometimes appear on the face, arms, and legs. Linear scleroderma is characterized by a single line or band of thickened and/or abnormally colored skin. The line typically runs down an arm or leg, but is sometimes observed across or down the forehead.

Systemic scleroderma refers to the form of the disease that includes the skin, as well as other tissues. Systemic sclerosis is classified as limited cutaneous scleroderma and diffuse cutaneous scleroderma.

Limited cutaneous scleroderma typically affects the skin only in certain areas: the fingers, hands, face, lower arms, and legs. Most people with limited disease have Raynaud's phenomenon, telangiectasias, and calcinosis. Calcinosis refers to the formation of calcium deposits in the connective tissues. When the deposits break through the skin, painful ulcers can result. Raynaud's phenomenon is a condition in which the small blood vessels of the hands and/or feet contract in response to cold or anxiety. As the vessels contract, the hands or feet turn white and cold, then blue. As blood flow returns, they become red. Fingertip tissues may suffer damage, leading to ulcers, scars, or gangrene. Telangiectasia is a condition caused by the swelling of tiny blood vessels, in which small red spots appear on the hands and face. While not painful, these red spots can be embarrassing or even disfiguring.

Diffuse cutaneous scleroderma is usually characterized by skin thickening that begins in the hands and spreads over much of the body. The hands, face, upper arms, upper legs, chest, and stomach can be affected, typically in a symmetrical fashion. Internally, it can damage vital organs. Patients often report a lose appetite and weight, fatigue, and joint swelling, and/or pain. Skin changes can cause the skin to swell, appear shiny, and feel tight and itchy.

The methods of the present invention can be used in the treatment of sclerodemma. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating scleroderma in a patient in need thereof. By “treating” is meant to include decreasing or eliminating one or more symptoms of scleroderma. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to both the skin (whether or not also effected) and to the underlying, effected, connective tissue. Over the course of one or more treatments, the ultrasound energy decreases or eliminates the presence, severity, and/or frequency of the symptoms of scleroderma by, for example, decreasing the local inflammatory response.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, skin irritation, rash, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decrease skin involvement, decreased reliance on pain or other medication, improvement in patient self-assessment of pain, quality of life, or other indicia.

### Still's Disease

Still's disease is a form of juvenile rheumatoid arthritis that, although more common in children, can also occur in adults.

Still's disease tends to effect multiple joints. Symptoms include joint pain, fatigue, sore throat, and high fever that falls and rises quickly. A non-itchy salmon colored skin rash is also a common symptom of the disease.

Current treatment for Still's disease includes the use of medications intended to decrease pain and inflammation. Such medications include non-steroidal anti-inflammatory medicines, as well as steroidal medications. Treatment often includes exercise and physical therapy to help preserve range of motion and help slow joint deterioration.

The methods of the present invention can be used in the treatment of Still's disease. Depending on the severity of the symptoms, more treatments and/or longer treatment times (time of each treatment) may be needed to produce the desired therapeutic efficacy. Note, however, although dramatic improvement may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating Still's disease in a patient in need thereof. By “treating” is meant to include decreasing or eliminating one or more symptoms of Still's disease. Low frequency ultrasound energy is administered (with or without a liquid spray) to effected tissue of the patient. The low frequency ultrasound energy is administered without contact between the skin overlying the effected tissue and the ultrasound transducer or other components of the device (non-contact distance). The low frequency ultrasound energy penetrates the skin to provide a therapeutic effect to the underlying, effected, connective tissue. Over the course of one or more treatments, the ultrasound energy decreases or eliminates the presence, severity, and/or frequency of the symptoms of Still's disease.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

Exemplary symptoms that can be treated include, but are not limited to, pain (including joint pain), swelling, fever, loss of range of motion, and decreased ability to perform daily tasks. Improvement in any of these symptoms can be measured by, for example, decrease in the number of swollen joints, decrease in the number of painful joints, increased range of motion, increased ability to perform daily tasks, decreased reliance on pain or other medication, improvement in patient self-assessment of pain, quality of life, or other indicia.

The above conditions are exemplary of the inflammatory disorders that can be treated using non-contact, low frequency ultrasound therapy. Any of the features of the methods described herein can be used in the treatment of any of the rheumatoid diseases or conditions described herein. In other words, low frequency ultrasound therapy (with or without the use of a liquid spray) can be used in the treatment of any inflammatory disorder, for example the rheumatoid disorders described herein.

### (v) Combination Therapy

In certain embodiments, the use of non-contact ultrasound (with or without liquid spray) is used to treat an inflammatory disorder (e.g., to decrease the symptoms of the inflammatory disorder) and is used to decrease or avoid the need for using traditional topical, injectable, or oral agents. For example, by using non-contact ultrasound therapy, the need for treatment using steroids, antibiotics, anti-inflammatories, astringents, etc., can be decreased or eliminated.

In other embodiments, however, non-contact ultrasound therapy is used in combination with other treatment modalities as part of a therapeutic regimen for treating an inflammatory disorder. When used in this way, non-contact ultrasound therapy can act additively or synergistically with other treatments. Exemplary therapies include, but are not limited to, antibiotics, hydrocortisone creams, benzoil peroxide, retinoids and other vitamin A based agents, steroids or other immunosuppressive agents (methotrexate, cyclosporin), and the like. Further exemplary therapies include cytokine antagonists, such as TNF-α antagonists designed to decrease expression of TNF-α. Further exemplary therapies include phototherapy, a specialized dietary regimen, acupuncture, stress management, exercise, and the like.

Some of the available therapies for inflammatory disorders have significant side-effects. One potential benefit of ultrasound therapy is the avoidance of administering drugs such as steroids, systemic anti-inflammatory agents, or immunosuppressive agents. However, for certain conditions or patients, drug therapy may still be needed to provide additional therapeutic benefit and, when needed, can be used in combination with ultrasound therapy. In certain embodiments, ultrasound therapy decreases the patient's dependence on drug therapy. In other words, the additive or synergistic effects of ultrasound energy and the administered medicament allow the achievement of the same or better therapeutic efficacy at a decreased dose of drug.

In certain embodiments, the use of ultrasound therapy, alone or in combination with drug, decreases a patient's reliance on pain medication. A decrease in reliance on pain medication can be assessed by measuring a decrease in the amount or frequency with which pain medication is consumed, as well as by assessing the frequency with which the patient requests additional pain medication.

When drug therapy is used in combination with ultrasound, the invention contemplates that the drug itself may be delivered via the ultrasound device. In other words, the liquid drug is delivered to an ultrasound transducer to generate a liquid spray, and the liquid spray and ultrasound energy are delivered to the patient and penetrate the patient's tissue. The invention also contemplates that the medicament can be topically applied directly to the patient tissue, and ultrasound energy (with or without an inert or medicated liquid spray) can be delivered to the topically applied medicament and to the underlying patient tissue. When used in this manner, the ultrasound energy facilitates the penetration of the drug into the patient's tissue. Additionally, the invention contemplates embodiments in which the medicament is administered as per its labeling instructions (e.g., topically, orally, intravenously, etc.) and ultrasound energy is delivered in a separate step prior to or following administration of the medicament.

In other embodiments, the drug is delivered as part of a therapeutic regimen, but ultrasound energy is not used to facilitate delivery.

Regardless of the mechanism by which drug is delivered, the invention contemplates combinatorial therapies that involve administering a drug as part of the therapeutic regimen. Exemplary drugs include, but are not limited to, corticosteroids, analgesics, non-steroidal anti-inflammatory agents, vitamin A derivatives, vitamin D derivatives, anti-mitotic agents, TNF-α inhibitory agents, antibiotics, anti-fungals, and immunosuppressants.

In other embodiments, however, non-contact ultrasound therapy is used in combination with other available treatments as part of a therapeutic regimen for treating an inflammatory disorder. When used in this way, non-contact ultrasound therapy can act additively or synergistically with other treatment modalities. Exemplary therapies include, but are not limited to, antibiotics, hydrocortisone creams or injections, steroids or other immunosuppressive agents (methotrexate, cyclosporin), and the like. Further exemplary therapies include phototherapy, a specialized dietary regimen, acupuncture, stress management, occupational therapy, physical therapy, and the like.

### (vi) Animal Models

The use of low frequency ultrasonic energy (in the presence or absence of liquid spray; in the presence or absence of additional therapeutic modalities) to treat one or more symptoms of an inflammatory disorder, particularly a rheumatoid disorder, can be tested in one or more animal models. Exemplary animal models are described briefly herein. However, numerous animal models exist and any model available in the art can be readily used to evaluate a particular treatment regimen (e.g., to evaluate number of treatment, duration of treatment, combination with one or more current treatment modalities, etc).

Numerous animal models for rheumatoid arthritis exist in the art. By way of non-limiting example, BioMedCode Hellas SA makes animal models for inflammatory conditions. Many of the company's models are approved by the FDA for testing potential treatments for RA. Their models include Tg197 and Tg5433 mice.

Additionally animal models of arthritis and rheumatoid arthritis include, but are not limited to, the models described in the following publications: Hammer et al., 1990, Cell 63: 1099-1112; Haqqi et al., 1992, PNAS 89: 1253-1255; Keffer et al., 1991, EMBO Journal 10: 4025-4031; Pelletier et al., 1997, Arthritis Rheum 40: 1012-1029; Trentham et al., 1977, Journal of Experimental Medicine 146: 857-868; Wooley et al., 1981, Journal of Experimental Medicine 154: 688-700. Recently, Bina and Wilder reviewed numerous available animal models for Rheumatoid arthritis. Bina and Wilder, 1999, Molecular Medicine Today 5: 367-369.

The carrageenin model for induced inflammation is often used to model conditions that have a significant inflammatory component. Injection of carrageenin into, for example, the paw of a dog, rabbit, rat, mouse, or other animal induces edema and is used as a model for inflammation. An example using dogs as a model is provided in Brooks et al., 1991, Pharmacol Methods 25: 275-283. This type of model can be used to assess efficacy of treatment on any of a number of inflammatory conditions.

Numerous models of scleroderma exist and can be used. Yamamoto recently summarized multiple animals models including the bleomycin-induced scleroderma model, the tight skin and tsk2 mouse, and the UCD chicken. Yamamoto, 2005, Current Rheumatology Review 1: 101-109.

Therapeutic regimens including one or more treatments with low frequency ultrasonic energy (alone or in combination with one or more additional treatment modalities) can be tested in one or more animal models. Exemplary models are described herein, although numerous additional animal models are well known in the art and can be similarly used. Treatment with ultrasonic energy is compared to, for example, no treatment controls or control treatment with one or more current, non-ultrasonic therapies. Additionally or alternatively, such models can be used to assess, for example, the effectiveness of a therapeutic regimen in which the frequency of treatments and/or the duration of each treatment are varied. Similarly, such models can be used to assess, for example, the effectiveness of a therapeutic regimen in which ultrasonic energy is delivered in the presence versus the absence of a fluid spray, as well as in the presence or the absence of a medicament (e.g., a fluid spray containing medicament and/or a topically applied medicament and/or a systemically administered medicament).

Additionally, therapeutic regimens including one or more treatments with low frequency ultrasonic energy (alone or in combination with one or more additional treatment modalities) can be tested in in vitro models (e.g., cell-based models, organ culture models). Further, such therapeutic regimens can be tested in vivo in human patients.

### (vii) Diagnostic Methods

One aspect of the present invention is based on the recognition that low frequency ultrasound can be used to decrease the inflammatory response by decreasing expression and/or activation of pro-inflammatory cytokines. As such, one aspect of the invention provides methods for decreasing expression and/or activation of one or more pro-inflammatory cytokines. The method can be performed on cells or tissue explants cultured or otherwise maintained in vitro. In such in vitro embodiments, cells or tissue explants in culture are contacted with low frequency ultrasound energy from a non-contact distance, as described throughout the application. The cells or tissue explants can be assessed to evaluate the decrease in expression and/or activation of one or more pro-inflammatory cytokines in comparison to untreated control. Exemplary pro-inflammatory cytokines that can be evaluated include, but are not limited to, TNFα, IL-1β, IL-8, p38 MAPK, other pro-inflammatory interleukins, and the like.

Suitable diagnostics methods can also be performed following in vivo treatment of tissues. Note that in this context the terms “in vitro” and “in vivo” are used to characterize the cells at the time of receiving the ultrasound treatment. Following treatment, the cells can be evaluated either in the context of the patient or animal or using an in vitro assay. The post-treatment evaluation method does not alter whether the ultrasound delivery occurred in vivo or in vitro.

In certain embodiments, ultrasound energy is delivered to effected tissue of a patient in need thereof (delivered in vivo), and expression and/or activation of one or more pro-inflammatory cytokines is evaluated following treatment. Expression and/or activation of one or more pro-inflammatory cytokines can be evaluated at any one or more time points following one or more treatments, and compared to expression and/or activation prior to initiation of treatment (but after the onset of symptoms of the inflammatory disorder). When used in this way, decrease in the local inflammatory response, as assessed by expression and/or activation of one or more pro-inflammatory cytokines, can be used to evaluate the progress of the treatment.

As indicated above, although the ultrasound energy is delivered in vivo, analysis of the one or more pro-inflammatory cytokines can be conducted in vivo or in vitro. For example, for in vitro analysis, suitable tissue samples can be taken over time and analyzed in vitro. In the case of an inflammatory disorder, small samples of synovial fluid can be taken for analysis. For in vivo analysis, vital dyes and agents can be used to help assess the inflammatory response in the tissue in its in vivo context and without the need to obtain a sample or biopsy from the patient. Regardless of whether the diagnostic step is conducted in vitro or in vivo, exemplary pro-inflammatory cytokines that can be evaluated include, but are not limited to, TNFα, IL-1β, IL-8, p38, other pro-inflammatory interleukins, and the like.

In certain embodiments, the diagnostic step is conducted multiple times throughout the course of treatment. In certain embodiments, the one or more diagnostic steps are used by a health care provider to help determine the duration of treatment, as well as whether ultrasound therapy should be used alone or combined with other therapies.

### EXEMPLIFICATIONS

The invention now being generally described, it will be more readily understood by reference to the following examples, which are included merely for purposes of illustration of certain aspects and embodiments of the present invention, and are not intended to limit the invention.

### Example 1

### Low Frequency Ultrasound does not Decrease Viability of a Human Monocyte-Derived Cell Line Cultured In Vitro

A human monocyte-derived cell line (THP-1) was used to evaluate the ability of low frequency ultrasound to modulate an inflammatory response. Before evaluating whether low frequency ultrasound decreased an inflammatory response, we assessed whether the ultrasound energy altered cell viability.

THP-1 cells were cultured in RPMI 1640 medium supplemented with fetal calf serum (FCS; 10% v/v), L-glutamine (2 mM), penicillin (100 U/ml), streptomycin (100 ug/ml), and sodium pyruvate (1 mM). The cells were maintained at 37° C. and 5% CO₂.

2-4 ml of THP-1 cell suspension was prepared and exposed to 3 minutes of either low frequency ultrasound delivered by the MIST™ Therapy system (Mist; manufactured by Celleration, Inc.; www.celleration.com), or to kinetic energy delivered by a nebulizer (KE). A third sample was not exposed to any treatment (untreated). Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium (e.g., “dry”). However, the energy could have been delivered via a fluid spray (e.g., saline solution, water, etc.).

Following treatment, the viability of the THP-1 cells was assessed by determining the percentage of live cells in the sample following treatment. As depicted in FIG. 1, we consistently observed greater than 90% cell survival in all three groups (untreated group=left most bar; ultrasound energy group (Mist)=center bar; kinetic energy group (KE)=right most bar). Thus, viability of these cells was not significantly affected by treatment with low frequency ultrasound.

To confirm that any negative effect on cell viability was not delayed, the number and viability of treated THP-1 cells was assessed both immediately after treatment, and following an additional 18 hours of post-treatment culture. The total number and viability of the cells did not differ, thus supporting the conclusion that the ultrasound therapy treatment had no immediate or delayed cytotoxic effects on cell viability.

### Example 2

### Low Frequency Ultrasound Specifically Inhibited an Inflammatory Response

TNFα is a proinflammatory cytokine that is upregulated as part of the inflammatory response. We evaluated whether low frequency ultrasound could reduce the inflammatory response by assessing TNFα production in response to LPS stimulation.

THP-1 cell suspensions were prepared and exposed to 3 minutes of either low frequency ultrasound delivered by the MIST™ Therapy system, or to kinetic energy delivered by a nebulizer. A third sample was not exposed to any treatment. Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium (“dry”). However, the energy could have been delivered via a fluid spray (e.g., saline solution, water, etc.).

Following treatment, each group of cells was cultured in 96 well-plates and stimulated with LPS (LPS from E. coli, Sigma-Aldrich) to induce an inflammatory reaction. Cells were plated at concentrations of 1×10⁶ cells/ml or 0.5×10⁶ cells/ml. Cells were stimulated with either 10 or 100 ng/ml LPS. Following 5 hours of LPS stimulation, TNFα protein concentration in the cell culture supernatants was determined using a commercially available ELISA kit (R&D Systems).

The cells treated with low frequency ultrasound produced less TNFα following LPS stimulation than the untreated cells or the cells exposed to kinetic energy. As depicted in FIG. 2, low frequency ultrasound treatment inhibited TNFα production by LPS stimulated THP-1 cells in comparison to that of untreated cells or cells treated with kinetic energy. In FIG. 2, TNFα was measured by ELISA and the percentage of the OD of the treated cells versus the untreated cells is given on the y-axis (untreated group=left most bar; ultrasound energy group (Mist)=center bar; kinetic energy group (KE)=right most bar). Thus, low frequency ultrasound treatment inhibited the inflammatory response in these cells.

To confirm that delivery of ultrasound energy in combination with a liquid spray (“wet”) had a similar effect, the above experiment was repeated using the MIST system to deliver ultrasound energy and a saline spray. Briefly, 2-4 ml of THP-1 cell suspension was placed in a Petri dish and subjected to Mist ultrasound therapy with saline solution. The saline solution is delivered to the ultrasound transducer which produces an atomized spray of saline. Ultrasound energy and the spray of saline is thus delivered to the cells. Following the treatment time (in this experiment—3 minutes), the cell suspension was collected and processed, as described above for cells treated “dry”.

Treatment of the THP-1 cells with ultrasound energy and a saline spray (“wet”) had a similar effect on LPS-induced TNF-α production as the “dry” treatment. Both treatments resulted in inhibition of LPS-induced TNF-α production in comparison to untreated and KE (non-ultrasound) controls.

### Example 3

### Low Frequency Ultrasound Specifically Inhibited an Inflammatory Response

p38 is typically upregulated as part of the inflammatory response. We evaluated whether low frequency ultrasound could reduce the inflammatory response by assessing p38 activation following LPS stimulation. Activation of p38 is often evaluated by detecting p38 phosphorylation using, for example, an antibody that recognizes phosphorylated p38.

THP-1 cell suspensions were prepared and exposed to 3 minutes of either low frequency ultrasound delivered by the MIST™ Therapy system, or to kinetic energy delivered by a nebulizer. A third sample was not exposed to any treatment. Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium. However, the energy could have been delivered via a fluid spray (e.g., saline solution, water, etc.).

Following treatment, each group of cells was cultured in 6 well-plates and stimulated with LPS (LPS from E. coli, Sigma-Aldrich) to induce an inflammatory reaction. Cells were stimulated with either 100 ng/ml LPS (or with 0 ng LPS as an unstimulated control). Following 30 minutes of LPS stimulation, the cells were lysed in Laemmli buffer at a concentration of 20×10⁶ cell/ml. Cell lysates (10 ul) were separated electrophoretically, and analyzed by Western blot using an antibody to phospho-p38 MAPK (Thr180/Tyr182) primary antibody and an HRP-conjugated sheep anti-rabbit secondary antibody (Amersham). Protein detection was performed by chemiluminescence with ECL Plus Western Blotting Detection (Amersham).

Treatment with low frequency ultrasound inhibited p38 activation (decreased the amount of phosphorylated p38 detected in the assay). As depicted in FIGS. 3 and 4, low frequency ultrasound treatment inhibited p38 activation in LPS stimulated THP-1 cells in comparison to that of untreated cells or cells treated with kinetic energy. FIG. 3 shows Western blot analysis using anti-phosphorylated p38 antibody and FIG. 4 provides a quantitative analysis of the intensity of the band shown in FIG. 3. As can be seen from FIGS. 3 and 4, treatment with low frequency ultrasound inhibited p38 activation in LPS stimulated cells. Thus, low frequency ultrasound treatment inhibited the inflammatory response in these cells.

### Example 4

### Low Frequency Ultrasound Specifically Inhibited an Inflammatory Response

The experiment outlined above in Example 3 was repeated. However, in this experiment, activation of both p38 and hsp27 were evaluated by evaluating the phosphorylation of p38 and hsp27. Briefly, THP-1 cells were subjected to 3 min of MIST, control treatment, or were untreated. Phosphorylation of p38 and hsp27 in ±LPS treated cells was assessed by western blot analysis. As shown in FIG. 5, low frequency ultrasound treatment attenuated activation of p38 and hsp27.

### Example 5

### The Effect of Low Frequency Ultrasound on the Inflammatory Response was Dependent on Treatment Time

The period of exposure to the low frequency ultrasound (the treatment time) corresponds to the amount of energy delivered. As such, a longer treatment time results in delivery of more energy. We evaluated whether Mist treatment would have a larger effect on cells if used for a longer treatment time.

THP-1 cell suspensions were prepared and exposed to either 3 or 6 minutes of low frequency ultrasound delivered by the MIST™ Therapy system (“dry”). Control samples treated with kinetic energy delivered by a nebulizer (for 3 or 6 minutes) or left untreated were also evaluated. As before, the cells exposed to low frequency ultrasound were treated at a non-contact distance from the cells and from their culture medium. In other words, the low frequency ultrasound was delivered at a non-contact distance from the cells and their culture medium.

Following treatment, each group of cells was cultured in 96 well-plates and stimulated with LPS (LPS from E. coli, Sigma-Aldrich) to induce an inflammatory reaction. Cells were plated at concentrations of 1×10⁶ cells/ml or 0.5×10⁶ cells/ml. Cells were stimulated with either 10 or 100 ng/ml LPS. Following 5 hours of LPS stimulation, TNFα protein concentration in the cell culture supernatants was determined using a commercially available ELISA kit (R&D Systems).

This experiment indicated that the anti-inflammatory response was proportional to the length of treatment, across this range of treatment times. Specifically, TNF-α production in LPS stimulated cells was approximately 45% that of controls following 3 minutes of Mist treatment, but only approximately 25% that of controls following 6 minutes of Mist treatment. Thus, the larger amount of ultrasound energy delivered over the longer treatment time had a noticeable impact on TNF-α production.

### REFERENCES

Clark (1996). The Molecular and Cellular Biology of Wound Repair, New York, N.Y., Plenum, pages 3-50.
Janeway and Medzhitov (2002). Annual Review of Immunology 20: 197-216.
Dong et al. (2002) Annual Review of Immunology 20: 55-72.

### INCORPORATION BY REFERENCE

All publications and patents mentioned herein, are hereby incorporated by reference in their entirety as if each individual publication or patent was specifically and individually indicated to be incorporated by reference.

### EQUIVALENTS

Those skilled in the art will know or be able to ascertain using no more than routine experimentation, many equivalents to the embodiments and practices described herein. Accordingly, it will be understood that the foregoing descriptions are to be considered in all respects illustrative, rather than limiting, of the invention. For example, a variety of systems and/or methods may be implemented based on the disclosure and still fall within the scope of the invention. The specifications and other disclosures in the patents, patent applications, and other references cited herein are hereby incorporated by reference in their entirety. Further, the invention contemplates combinations of any of the foregoing aspects and embodiments of the invention.

1. A method of treating an inflammatory disorder in a patient in need thereof, comprising delivering low frequency ultrasound energy from a non-contact distance to effected tissue of the patient in need thereof, wherein said low frequency ultrasound energy penetrates skin overlying the effected tissue to provide a therapeutic effect to decrease one or more symptoms of the inflammatory disorder, and wherein the inflammatory disorder is a rheumatoid disorder.

2. The method of claim 1, wherein the rheumatoid disorder is selected from one or more of rheumatoid arthritis, juvenile arthritis, bursitis, spondylitis, gout, scleroderma, Still's disease, or vasculitis.

3-9. (canceled)

10. The method of claim 1, wherein the effected tissue is selected from one or more of joints, tendons, ligaments, or blood vessels.

11. The method of claim 10, wherein the effected tissue is one or more joints.

12. (canceled)

13. The method of claim 1, wherein the low frequency ultrasound energy is delivered via a liquid spray, and the method comprises delivering the low frequency ultrasound energy and the liquid spray from a non-contact distance to effected tissue of the patient in need thereof to provide a therapeutic effect.

14. The method of claim 1, wherein the low frequency ultrasound energy is delivered in the absence of a liquid spray or coupling agent.

15-17. (canceled)

18. A method for reducing pain associated with a rheumatoid disorder in a patient in need thereof, comprising
providing a transducer which can emit low frequency ultrasonic energy;
delivery said low frequency ultrasonic energy to effected tissue of said patient;
wherein said ultrasonic energy is delivered from a non-contact distance between a vibrating tip of the transducer and said effected tissue of said patient, and wherein the delivered ultrasonic energy provides a therapeutic effect to reduce pain associated with said rheumatoid disorder.

19. The method of claim 18, wherein the effected tissue is selected from one or more of joints, tendons, and ligaments.

20. The method of claim 18, wherein the ultrasonic energy is delivered via a liquid spray, and the method comprises delivering the low frequency ultrasonic energy and the liquid spray to said patient.

21. The method of claim 20, wherein the liquid spray is generated by delivering liquid to a distal portion of the transducer.

22. The method of claim 18, wherein the ultrasonic energy is delivered in the absence of a liquid spray or coupling agent.

23. The method of claim 18, wherein the method is part of a therapeutic regimen combining one or more additional treatment modalities.

24. The method of claim 23, wherein the one or more additional treatment modalities comprises applying a topical medicament to the effected tissue or administering a systemic medicament prior to and/or following delivering said ultrasonic energy.

25. The method of claim 23, wherein the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

26. The method of claim 18, wherein delivering ultrasonic energy comprises delivering ultrasonic energy for at least about 2 consecutive minutes.

27. The method of claim 18, wherein the method comprises delivering ultrasonic energy at least twice per week for at least two weeks.

28. The method of claim 18, wherein the rheumatoid disorder is selected from rheumatoid arthritis or juvenile arthritis.

29. The method of claim 18, wherein the rheumatoid disorder is selected from any of bursitis, spondylitis, gout, scleroderma, Still's disease, and vasculitis.

30. A method for treating a rheumatoid disorder, comprising
providing a transducer which can emit low frequency ultrasonic energy;
delivering said ultrasonic energy to a patient in need thereof;
wherein said ultrasonic energy is delivered from a non-contact distance between a vibrating tip of the transducer and treated patient tissue, and wherein the delivered ultrasonic energy provides a therapeutic effect to decrease one or more symptoms of said rheumatoid disorder in said patient.

31. The method of claim 30, wherein the ultrasonic energy is delivered to at least one of said patient's joints, ligaments, or tendons.

32. The method of claim 30, wherein the ultrasonic energy penetrates the patient tissue to provide a therapeutic effect.

33. The method of claim 30, wherein the ultrasonic energy is delivered via a liquid spray, and the method comprises delivering the low frequency ultrasonic energy and the liquid spray to the patient.

34. The method of claim 33, wherein the liquid spray is generated by delivering liquid to a distal portion of the transducer.

35. The method of claim 30, wherein the ultrasonic energy is delivered in the absence of a liquid spray or coupling agent.

36. The method of claim 30, wherein the method is part of a therapeutic regimen combining one or more additional treatment modalities.

37. The method of claim 36, wherein the one or more additional treatment modalities comprises applying a topical medicament to the treated tissue or systemically administering medicament prior to and/or following delivering said ultrasonic energy.

38. The method of claim 36, wherein the one or more additional treatment modalities comprises a dietary regimen, an exercise regimen, yoga, heat, cold, acupuncture, acupressure, oral or intravenous analgesics, anti-inflammatory agents, corticosteroids, or anti-TNFα therapeutic agents.

39. The method of claim 30, wherein delivering ultrasonic energy comprises delivering ultrasonic energy for at least about 2 consecutive minutes.

40. The method of claim 30, wherein the method comprises delivering ultrasonic energy at least twice per week for at least two weeks.

41. The method of claim 30, wherein the one or more symptoms are selected from one or more of inflammation, pain, tingling, weakness, decreased grip strength, swelling, itchiness, burning, fever, and decreased range of motion.

42. The method of claim 30, 35, wherein the rheumatoid disorder is selected from rheumatoid arthritis or juvenile arthritis.

43. The method of claim 30, wherein the rheumatoid disorder is selected from any of bursitis, spondylitis, gout, scleroderma, Still's disease, and vasculitis.

44. A method for managing symptoms of a rheumatoid disorder, comprising
providing a transducer which can emit low frequency ultrasonic energy;
delivering said ultrasonic energy to a patient in need thereof for at least two consecutive minutes at least twice per week;
wherein said ultrasonic energy is delivered from a non-contact distance between a vibrating tip of the transducer and treated patient tissue, and wherein the delivered ultrasonic energy provides a therapeutic effect to treat one or more symptoms of said rheumatoid disorder.

45. A method for decreasing the number of painful or swollen joints in a patient suffering from a rheumatoid disorder, comprising
providing a transducer which can emit low frequency ultrasonic energy;
delivering said ultrasonic energy to said patient;
wherein said ultrasonic energy is delivered from a non-contact distance between a vibrating tip of the transducer and treated patient tissue, and wherein the delivered ultrasonic energy provides a therapeutic effect to decrease the number of painful or swollen joints in said patient.

46. A method for decreasing expression of an inflammatory cytokine in a patient having a rheumatoid disorder, comprising
providing a transducer which can emit low frequency ultrasonic energy;
delivering said ultrasonic energy to said patient in need thereof;
wherein the delivered ultrasonic energy decreases expression of said inflammatory cytokine in one or more of an effected joint, ligament, tendon, skin, or blood vessel of said patient, and wherein said inflammatory cytokine is TNFα.

47. The method of claim 46, wherein the ultrasonic energy is delivered via a liquid spray, and the method comprises delivering the low frequency ultrasonic energy and the liquid spray to the patient.

48. The method of claim 47, wherein the liquid spray is generated by delivering liquid to a distal portion of the transducer.

49. The method of claim 46, wherein the ultrasonic energy is delivered in the absence of a liquid spray or coupling agent.

50. The method of claim 30, wherein the ultrasonic energy is delivered at a frequency of approximately 200 kHz to 400 kHz.

51. The method of claim 30, wherein the ultrasonic energy is delivered at a frequency of approximately 20 kHz to 200 kHz.

52. The method of claim 30, wherein the ultrasonic energy is delivered at a frequency of approximately 30-50 kHz.

53. The method of claim 30, wherein the ultrasonic energy is delivered at a frequency of approximately 40 kHz.

54. The method of claim 30, wherein the ultrasonic energy level provided to patient tissue is approximately 0.1-2.0 watts/cm².

55. The method of claim 30, wherein the ultrasonic energy level provided to patient tissue is approximately 0.1-1.0 watts/cm².

56. The method of claim 30, wherein the ultrasonic energy level provided to patient tissue is approximately 0.1-0.7 watts/cm².

57. The method of claim 30, wherein the ultrasonic energy is delivered with a liquid spray, and wherein the liquid spray is selected from a saline solution or other substantially inert liquid.

58. The method of claim 30, wherein the ultrasonic energy is delivered with a liquid spray, and wherein the liquid spray includes a therapeutic medicament.
